SEQUENCE LISTING (21 sequences)

SEQ ID NO: 1 - DNA, 8 bp, Artificial
  RNA splicing donor site sequence
gtaagagk

SEQ ID NO: 2 - DNA, 10 bp, Artificial
  RNA splicing acceptor site sequence
gwkscyrcag

SEQ ID NO: 3 - DNA, 7 bp, Artificial
  branch-point sequence
tactway

SEQ ID NO: 4 - DNA, 41 bp, Artificial
  SpRNAi N1-sense sequence
gtaagaggatccgatcgcaggagcgcaccatcttcttcaag

SEQ ID NO: 5 - DNA, 46 bp, Artificial
  SpRNAi N1-antisense sequence
cgcgtcttgaagaagatggtgcgctcctgcgatcggatcctcttac

SEQ ID NO: 6 - DNA, 42 bp, Artificial
  SpRNAi N2-sense sequence
gtaagaggatccgatcgcttgaagaagatggtgcgctcctga

SEQ ID NO: 7 - DNA, 46 bp, Artificial
  SpRNAi N2-antisense sequence
cgcgtcaggagcgcaccatcttcttcaagcgatcggatcctcttac

SEQ ID NO: 8 - DNA, 70 bp, Artificial
  SpRNAi N3-sense sequence
gtaagaggatccgatcgcaggagcgcaccatcttcttcaagttaacttgaagaagatggtgcgctcctga

SEQ ID NO: 9 - DNA, 74 bp, Artificial
  SpRNAi N3-antisense sequence
cgcgtcaggagcgcaccatcttcttcaagttaacttgaagaagatggtgcgctcctgcgatcggatcctcttac

SEQ ID NO: 10 - DNA, 47 bp, Artificial
  SpRNAi N4-sense sequence
cgcgttactaactggtacctcttctttttttttttgatatcctgcag

SEQ ID NO: 11 - DNA, 45 bp, Artificial
  SpRNAi N4-antisense sequence
gtcctgcaggatatcaaaaaaaaaagaagaggtaccagttagtaa

SEQ ID NO: 12 - DNA, 689 bp, Heteractis crispa
  gene (1)..(689)
atggtgagcggcctgctgaaggagagtatgcgcatcaagatgtacatggagggcaccgtgaacggccactacttcaagtgcgagggcgagggcgacggcaaccccttcgccggcacccagagcatgagaatccacgtgaccgagggcgcccccctgcccttcgccttcgacatcctggccccctgctgcgagtacggcagcaggacgaccttcgtgcaccacaccgccgagatccccgacttcttcaagcagagcttccccgagggcttcacctgggagagaaccaccacctacgaggacggcggcatcctgaccgcccaccaggacaccagcctggagggcaactgcctgatctacaaggtgaaggtgcacggcaccaacttccccgccgacggccccgtgatgaagaacaagagcggcggctgggagcccagcaccgaggtggtgtaccccgagaacggcgtgctgtgcggccggaacgtgatggccctgaaggtgggcgaccggcacctgatctgccaccactacaccagctaccggagcaagaaggccgtgcgcgccctgaccatgcccggcttccacttcaccgacatccggctccagatgctgcggaagaagaaggacgagtacttcgagctgtacgaggccagcgtggcccggtacagcgacctgcccgagaaggccaactg

SEQ ID NO: 13 - DNA, 27 bp, Artificial
  rGFP PCR-sense primer
ctcgagcatggtgagcggcctgctgaa

SEQ ID NO: 14 - DNA, 27 bp, Artificial
  rGFP PCR-antisense primer
tctagaagttggccttctcgggcaggt

SEQ ID NO: 15 - DNA, 25 bp, Artificial
  SpRNAi P1-sense sequence
cgcaagcagggccaaattgtgggta

SEQ ID NO: 16 - DNA, 31 bp, Artificial
  SpRNAi P1-antisense sequence
tagcacccacaatttggccctgcttgtgcgc

SEQ ID NO: 17 - DNA, 25 bp, Artificial
  SpRNAi P2-sense sequence
cgacccacaatttggccctgcttga

SEQ ID NO: 18 - DNA, 31 bp, Artificial
  SpRNAi P2-antisense sequence
tagccaagcagggccaaattgtgggttgcgc

SEQ ID NO: 19 - DNA, 51 bp, Artificial
  SpRNAi P3-sense sequence
cgcaagcagggccaaattgtgggtttaaacccacaatttggccctgcttga

SEQ ID NO: 20 - DNA, 57 bp, Artificial
  SpRNAi P3-antisense sequence
tagcacccacaatttggccctgcttgaattcaagcagggccaaattgtgggttgcgc

SEQ ID NO: 21 - DNA, 75 bp, Artificial
  Northern blot anti-rGFP/eGFP probe
cctggccccctgctgcgagtacggcagcaggacgtaagaggatccgatcgcaggagcgcaccatcttcttcaagt

(19) United States
(12) Patent Application Publication
(10) Pub. No.: US 2006/0228800 A1
(43) Pub. Date: Oct. 12, 2006

(54) Novel Transgenic Methods Using intronic RNA

(76) Inventors: Shi-Lung Lin, Arcadia, CA (US); Shao-Yao Ying, San Marino, CA (US)

Correspondence Address: Shi-Lung Lin;Shao-Yao Ying, 1953 Wellesley Road, San Marino, CA, 91108, US

(21) Appl. No.: 11/278,143

(22) Filed: Mar. 31, 2006

Related U.S. Application Data
(63) Continuation-in-part of application No. 10/439,262, filed on May 15, 2003.
(60) Provisional application No. 60/677,216, filed on May 2, 2005.

(51) Int. Cl. C12N 15/88; C12N 15/82; C12N 15/74

(52) U.S. Cl. 435/455; 435/471; 435/473; 435/458; 435/468

(57) ABSTRACT

The present invention relates to a method and composition for generating an artificial intron and its components capable of producing microRNA (miRNA) molecules and thus inducing specific gene silencing effects through intracellular RNA interference (RNAi) mechanisms, and the relative utilization thereof. The miRNA-producing intron so generated is not only useful for delivering desired miRNA function into the intron-mediated transgenic organisms or cells but also useful for suppressing unwanted gene function in the transgenic organisms or cells thereof. Furthermore, the derivative products of this novel man-made miRNA-producing intron have utilities in probing gene functions, validating drug targets, generating transgenic animals and gene-modified plants, developing anti-viral vaccines and treating as well preventing gene-related diseases (gene therapy).

### CLAIM OF THE PRIORITY

The present application claims priority to U.S. Provisional Application Ser. No. 60/677,216 filed on May 2, 2005, entitled “Novel Transgenic Animal Models Using RNA Interference” and the present application is a continuation-in-part application of the U.S. patent application Ser. No. 10/439,262 filed on May 15, 2003, entitled “RNA-Splicing and Processing-Directed Gene Silencing and the Relative Applications Thereof”, which are hereby incorporated by reference as if fully set forth herein.

### GOVERNMENT FUNDING

This invention was made with support in part by a grant from NIH (CA 85722). Therefore, the U.S. government has certain rights.

### FIELD OF THE INVENTION

This invention relates to a means for regulation of gene function. More particularly, the present invention relates to a method and composition for generating an artificial intron and its components capable of producing microRNA (miRNA) molecules via intracellular RNA splicing and/or processing mechanisms, and thus inducing transgenic gene silencing effects of RNA interference (RNAi) on the cell or cells of a targeted organism, and the relative utilities thereof. The miRNA-producing intron so generated is useful for not only delivering desired miRNA function but also suppressing unwanted gene activity in the intron-mediated transgenic organism.

### BACKGROUND OF THE INVENTION

Therapeutic intervention of a genetic disease can be achieved by regulating specific disease-associated genes, such as replacing impaired gene functions or suppressing unwanted gene functions. Plasmids and viral vectors are commonly used for introducing active genes into a cell to repair impaired gene functions. To suppress unwanted gene functions, antisense oligonucleotides (U.S. Pat. No. 6,066,500 to Bennett) and small molecule drugs are often used as therapeutic agents. With the advance of recent RNA interference (RNAi) technologies, novel small RNA agents have been developed to provide more efficient and less toxic means in gene regulation, including utilization of long double-stranded RNA (dsRNA) (U.S. Pat. No. 6,506,599 to Fire), double-stranded short interfering RNA (siRNA) (Elbashir et. al. (2001) Nature 411: 494-498) and DNA-RNA interfering molecules (D-RNAi) (Lin et. al. (2001) Biochem. Biophys. Res. Commun. 281: 639-644), which may have great industrial and therapeutic applications.

The mechanism of RNAi elicits post-transcriptional gene silencing (PTGS) phenomena capable of inhibiting specific gene functions with high potency at a few nanomolar dosage, which has been proven to be effective longer and much less toxic than traditional gene therapies using antisense oligonucleotides or small molecule drugs (Lin et. al. (2001) Current Cancer Drug Targets 1: 241-247). Based on prior studies, the siRNA-induced gene silencing effect usually lasts up to one week, while that of D-RNAi can sustain over one month. These phenomena appear to evoke an intracellular gene sequence-specific RNA degradation process, affecting all highly homologous gene transcripts, called co-suppression. It has been proposed that such a co-suppression effect results from the generation of small RNA products (21˜25 nucleotide bases) by enzymatic activities of RNA-directed RNA polymerases (RdRp) and/or endoribonucleases III (RNaseIII) on aberrant RNA templates, which are derived from foreign transgenes or viral infections (Grant, S. R. (1999) Cell 96: 303-306; Lin et. al. (2001) supra; Bartel, D. P. (2004) Cell 116: 281-297; Lin et. al. (2004a) Drug Design Reviews 1: 247-255).

Although RNAi phenomena appear to offer a new avenue for suppressing gene function, the applications thereof have not been demonstrated to work constantly and safely in higher vertebrates, including avian, mammal and human. For example, findings of the siRNA-mediated RNAi effect are based on the use of double-stranded RNA (dsRNA), which has shown to cause interferon-induced non-specific RNA degradation in vertebrates (Stark et. al. (1998) Annu. Rev. Biochem. 67: 227-264; Elbashir et. al. supra; U.S. Pat. No. 4,289,850 to Robinson; and U.S. Pat. No. 6,159,712 to Lau). Such an interferon-induced cytotoxic response usually reduces the specificity of RNAi-associated gene silencing effects and results in global, non-specific RNA degradation in cells (Stark et. al. supra; Elbashir et. al. supra). Especially in mammalian cells, it has been noted that the gene silencing effects of RNAi are disturbed when the siRNA size is longer than 25 base-pairs (bp). Although transfection of siRNA or small hairpin RNA (shRNA) sized less than 21 bp may overcome such a problem, unfortunately for transgenic and therapeutic use, this limitation in size impairs the usefulness of siRNA and shRNA because it is difficult to deliver such small and unstable RNA constructs in vivo due to the abundant RNase activities in higher vertebrates (Brantl S. (2002) Biochimica et Biophysica Acta 1575: 15-25).

With the advance of transgenic methods in gene delivery, a functional gene is preferably transfected into a cell or an organism, such as plant, animal and human being, using gene-expressing vector vehicles, including retroviral vector, lentiviral vector, adenoviral vector, adeno-associated viral (AAV) vector and so on. The desirable gene function so obtained in the cell and organism is activated through gene transcription and subsequently translation to form a functional polypeptide or protein for compensating a gene dysfunction or for competing with the homologous gene function. The main purpose of such a vector-based transgenic approach is to maintain long-term gene modulation under the control of cellular transcription and translation machineries. However, prior vector-based transgenic technologies, including antisense oligonucleotide and dominant-negative gene inhibitor vectors, have been shown to involve tedious works in target selection and have frequently resulted in inconsistent and unstable effectiveness (Jen et. al. (2000) Stem Cells 18: 307-319).

Recent utilization of siRNA-expressing vectors has improved transgenic stability and offered relatively long-term RNAi effects on vector-based gene modulation (Tuschl et. al. (2002) Nat Biotechnol. 20: 446-448). Although prior arts (Miyagishi et. al. (2002) Nat Biotechnol 20: 497-500; Lee et. al. (2002) Nat Biotechnol 20: 500-505; Paul et. al. (2002) Nat Biotechnol 20: 505-508) attempting to use this siRNA approach have succeeded in maintaining constant gene silencing efficacy, their strategies failed to provide a specific RNAi effect on a targeted cell population because of the use of ubiquitous type III RNA polymerase (Pol-III) promoters. Pol-III promoters, such as U6 and H1, are activated in almost all cell types, making tissue-specific gene targeting impossible. Further, because the read-through effect of Pol-III activity occurs on a short transcription template in the absence of proper termination, large RNA products longer than desired 18-25 bp can be synthesized and cause unexpected interferon cytotoxicity (Gunnery et. al. (1995) Mol Cell Biol. 15: 3597-3607; Schramm et. al. (2002) Genes Dev 16: 2593-2620). Such a problem can also result from the competitive conflict between the Pol-III promoter and another vector promoter (i.e. LTR and CMV promoters). Sledz et al. and us have found that high dosage of siRNA (e.g., >250 nM in human T cells) caused strong cytotoxicity similar to that of dsRNA (Sledz et. al. (2003) Nat Cell Biol. 5: 834-839; Lin et al. (2004b) Intrn'l J. Oncol. 24: 81-88). This toxicity is due to the double-stranded structures of siRNA and dsRNA, which activates the interferon-mediated non-specific RNA degradation and programmed cell death through signaling via the PKR and 2-5A systems (Stark et. al. supra). Interferon-induced protein kinase PKR triggers cell apoptosis, while activation of interferon-induced (2′,5′)-oligoadenylate synthetase (2-5A) system leads to extensive cleavage of single-stranded RNAs (i.e. mRNAs). Both PKR and 2-5A systems contain dsRNA-binding motifs which are sensitive to dsRNA and siRNA, but not to single-strand microRNA (miRNA) or RNA-DNA duplex. Thus, these disadvantages limit the use of Pol-III-based RNAi vector systems in vivo.

In sum, in order to improve the delivery stability, targeting specificity and transgenic safety of modern vector-based RNAi technologies in vivo, a better induction and maintenance strategy is highly desired. Therefore, there remains a need for an effective, stable and safe gene modulation method as well as agent composition for regulating targeted gene function via the novel RNAi and/or PTGS mechanisms.

### SUMMARY OF THE INVENTION

Research based on gene transcript (e.g. mRNA), an assembly of protein-coding exons, is fully described throughout the literature, taking the fate of spliced introns to be digested for granted (Clement et. al. (1999) RNA 5: 206-220; Nott et. al. (2003) RNA 9: 607-617). Is it true that the non-protein-coding intron is destined to be a metabolic waste without function or there is a function for it which has not yet been discovered? Recently, this misconception was corrected by the observation of intronic microRNA (miRNA). Intronic miRNA is a new class of small single-stranded regulatory RNAs derived from the processing of pre-mRNA introns. Approximately 10-30% of a spliced intron is exported into cytoplasm with a moderate half-life (Clement et. al. supra). miRNA is a single-stranded RNA molecule usually sized about 18-25 nucleotides (nt) in length and is capable of either directly degrading its intracellular messenger RNA (mRNA) target or suppressing the protein translation of its targeted mRNA, depending on the complementarity between the miRNA and its target. In this way, the intronic miRNA is functionally similar to previously described siRNA, but differs from them in the structural conformation and the requirement for Pol-II RNA transcription and splicing for its biogenesis (Lin et. al. (2003) Biochem Biophys Res Commun 310:754-760).

As shown in FIG. 1, the intronic miRNA biogenesis relies on the coupled interaction of nascent Pol-II-mediated pre-mRNA transcription and intron excision, occurring within certain nuclear regions proximal to genomic perichromatin fibrils (Lin et. al. (2004a) supra; Ghosh et. al. (2000) RNA 6: 1325-1334). In eukaryotes, protein-coding gene transcripts are produced by type-II RNA polymerases (Pol-II). The transcription of a genomic gene generates precursor messenger RNA (pre-mRNA), which contains four major parts including 5′-untranslated region (UTR), protein-coding exon, non-coding intron and 3′-UTR. Broadly speaking, both 5′- and 3′-UTR can be seen as a kind of intron extension. Introns occupy the largest proportion of non-coding sequences in the pre-mRNA. Each intron can be ranged up to thirty or so kilo-bases and is required to be excised out of the pre-mRNA content before mRNA maturation. This process of pre-mRNA excision and intron removal is called RNA splicing, which is executed by intracellular spliceosomes. After RNA splicing, some of the intron-derived RNA fragments are further processed to form microRNA (miRNA), which can effectively silence its targeted genes via an RNA interference (RNAi) mechanism, while exons of the pre-mRNA are ligated together to form a mature mRNA for protein synthesis.

Our present invention discloses a novel function of intron in the aspect of gene regulation and its relative utilities thereof. As shown in FIG. 2, based on the intracellular RNA splicing and intron processing mechanisms, we have designed a recombinant gene construct containing at least a splicing-competent intron (SpRNAi), which is able to inhibit the function of a gene that is partially or completely complementary to the intron sequence. After intron removal, the exons of the recombinant gene transcript will be linked together and become a mature mRNA molecule for protein synthesis. Without being bound by any particular theory, the method for generating and using the present invention relies on the genetic engineering of RNA splicing and processing apparatuses to form an artificial intron containing at least a desired RNA insert for miRNA production. The intron can be further incorporated into a gene for co-expression along with the gene transcript (pre-mRNA) in a cell or an organism. During mRNA maturation, the desired RNA insert will be released by RNA splicing and processing machineries and then triggers a desired gene silencing effect on genes and gene transcripts complementary to the RNA insert, while the exons of the recombinant gene transcript are linked together to form mature mRNA for expression of a desirable gene function, such as translation of a reporter protein selected from the group of green fluorescent protein (GFP), luciferase, lac-Z, and their derivative homologues. The expression of the reporter protein is useful for locating the production of desired intronic RNA molecules, facilitating splicing accuracy and preventing unwanted nonsense-mediated RNA degradation.

In accordance with the present invention, the mature RNA molecule formed by the linkage of exons may be useful in conventional gene therapy to replace impaired or missing gene function, or to increase specific gene expression. Additionally, the present invention provide novel compositions and means in producing intracellular gene silencing molecules by way of RNA splicing and processing mechanisms to elicit either an antisense oligonucleotide effect or an RNA interference (RNAi) effect useful for inhibiting gene function. The RNA splicing- and processing-generated gene silencing molecules, such as antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, resulting from the present invention is preferably used to target a gene selected from the group consisting of pathogenic nucleic acid, bacterial gene, viral gene, mutated gene, oncogene, jumping gene, transposon, microRNA gene and any other type of protein-coding as well as non-protein-coding genes.

In one preferred embodiment (FIG. 3), the present invention provides a method of using a novel composition for suppressing gene function or silencing gene(s), comprising the steps of: a) providing: i) a substrate expressing a targeted gene, and ii) an expression-competent composition comprising a recombinant gene capable of producing a specific RNA transcript, which is in turn able to generate pre-designed gene silencing molecules through intracellular RNA splicing and/or processing mechanisms to knock down the targeted gene expression or to suppress the targeted gene function in the substrate; b) treating the substrate with the composition under conditions such that the targeted gene function in the substrate is inhibited. The substrate can express the targeted gene either in cell, ex vivo or in vivo. In one aspect, the RNA splicing- and processing-generated gene silencing molecule is an RNA insert located within the intron of the recombinant gene and is capable of silencing a targeted gene selected from the group consisting of pathogenic nucleic acid, bacterial gene, viral gene, mutated gene, oncogene, diseased gene, jumping gene, transposon, matched miRNA gene and any other type of physiologically functional genes. Alternatively, such an RNA insert can also be artificially incorporated into the intron region of any kind of genes that are expressed in a cell or an organism. In principle, this kind of intronic insertion into a cellular gene can be accomplished using homologous recombination, transposon delivery, jumping gene integration and retroviral infection (as described in Examples 2-13 and FIGS. 3-16).

In another aspect, the recombinant gene of the present invention is constructed based on the natural pre-mRNA structure. The recombinant gene is consisted of two major different parts: exon and intron. The exon part is ligated after RNA splicing to form a functional mRNA and protein for tracking the release of the intronic RNA insert(s), while the intron part is spliced out of the recombinant gene transcript and further processed into a desired intronic RNA molecule, serving as the aforementioned antisense or RNAi molecule, including antisense RNA, miRNA, siRNA, shRNA and dsRNA, etc. These desired intronic RNA molecules may comprise at least a stem-loop structure containing a sequence domain homologous to (A/U)UCCAAGGGGG motifs, pre-miRNA loops or tRNA loops for accurate excision of the desired RNA molecule out of the intron and also for transporting the desired RNA molecule from nucleus to cytoplasm. The 5′-end of the intron contains a splicing donor site homologous to either GTAAGAGK or GU(A/G)AGU motifs, while its 3′-end is a splicing acceptor site that is homologous to either TACTWAY(N)mGWKSCYRCAG or CT(A/G)A(C/T)NG motifs, and preferably m≧1. The adenosine “A” nucleotide of the CT(A/G)A(C/T)NG sequence transcripts is part of (2′-5′)-linked branch-point acceptor formed by cellular (2′-5′)-oligoadenylate synthetases in eukaryotes, and the symbolic “N” nucleotide is either a nucleotide (e.g. deoxyadenosine, deoxyguanosine, deoxycytidine, deoxythymine, deoxyuridine, riboxyadenosine, riboxyguanosine, riboxycytidine, riboxythymine and riboxyuridine) or an oligonucleotide, most preferably a T- and/or C-rich oligonucleotide sequence. There could be a linker nucleotide sequence for the connection of the stem-loop to either a splicing donor or acceptor site, or both.

In another preferred embodiment of the present invention (FIGS. 4-6), the recombinant gene composition can be cloned into an expression-competent vector. The expression-competent vector is selected from a group consisting of plasmid, cosmid, phagemid, yeast artificial chromosome, transposon, jumping gene, retroviral vector, lentiviral vector, lambda vector, adenoviral (AMV) vector, adeno-associated viral (AAV) vector, modified hepatitis virus vector, cytomegalovirus (CMV)-related viral vector, and plant-associated mosaic virus, such as tabacco mosaic virus (TMV), tomato mosaic virus (ToMV), Cauliflower mosaic virus (CaMV) and poplar mosaic virus (PopMV). The strength of this strategy is in its deliverability through the use of vector transfection and viral infection, providing a stable and relatively long-term effect of specific gene silencing. Applications of the present invention include, without limitation, therapy by suppression of disease-related genes, vaccination directed against viral genes, treatment of microbe-related genes, genetic research of signal transduction pathways with systematic or specific knockdown of involved genes, and high throughput screening of gene functions in conjunction with microarray technologies, etc. The present invention can also be used as a tool for studying gene function in certain physiological and therapeutic conditions, providing a composition and method for altering the characteristics of an eukaryotic cell or organism. The cell or organism can be selected from the group of normal, pathogenic, cancerous, virus-infected, microbe-infected, physiologically diseased, genetically mutated, genetic engineering-modified microbes, cells, tissues, organs, plants, animals or humans.

In one aspect, the recombinant gene, for example, encoding an antisense RNA molecule as shown in FIG. 4, is generated by intracellular RNA splicing and processing mechanisms, ranged from a few to a few hundred ribonucleotides in length. Such an antisense RNA molecule elicits antisense gene knockdown activity for suppressing targeted gene function in cells. Alternatively, the antisense RNA molecule can bind to the sense strand of targeted gene transcripts to form long double-stranded RNA (dsRNA) for inducing interferon-associated cytotoxicity in order to kill the transfected cells, while the transfected cells is derived from a substrate organism selected from the group of cancerous, virus-infected, microbe-infected, physiologically diseased, genetically mutated or genetically engineering-modified, pathogenic plants or animals and so on. In another aspect, the present invention can be used in relation to posttranscriptional gene silencing (PTGS) technologies as a powerful new strategy in the field of gene therapy and transgenic model research (FIGS. 5-6). The present invention functioning via intracellular RNA splicing and/or processing mechanisms can produce RNAi molecules, such as small interfering RNA (siRNA), microRNA (miRNA) and small hairpin RNA (shRNA), or their combinations that are able to induce RNAi- and/or PTGS-like gene silencing phenomena. These RNAi molecules so obtained are of 12 to 38 nucleotides in length, preferably of 18 to 25 nucleotides. These RNAi molecules are desired to be produced intracellularly under the control of a gene-specific RNA promoter, such as type-II RNA polymerase (Pol-II) promoters and viral promoters. In plants, type-IV RNA polymerase (Pol-IV) promoters can also be used for the same purpose as Pol-II. The viral promoters include RNA promoters and their derivatives isolated from bacteriaphage (T7, SP6, M13), cytomegalovirus (CMV), retrovirus long-terminal region (LTR), hepatitis virus, adenovirus (AMV), adeno-associated virus (AAV), and plant-associated mosaic virus.

To produce small RNA molecules, such as siRNA, miRNA and shRNA, via RNA splicing and processing mechanisms, an expression-competent vector may be needed for stable transfection and expression of the intron-containing pre-mRNA molecule. The desired RNA molecule is produced intracellularly by promoter-driven mRNA transcription and then released by the RNA splicing and processing machineries. The expression-competent vector can be any nucleotide composition selected from a group consisting of plasmid, cosmid, phagemid, yeast artificial chromosome, transposon, jumping gene, retroviral vector, lentiviral vector, lambda vector, AMV, CMV, AAV, modified Hepatitis-virus vector, plant-associated mosaic viruses, and a combination thereof. The expression of the pre-mRNA is driven by either a viral or a cellular RNA polymerase promoter, or both. For example, a lentiviral or retrovirual LTR promoter is sufficient to provide up to 5×10⁵ copies of pre-mature mRNA per cell, while a CMV promoter can transcribe over 10⁶ to 10⁸ copies of pre-mature mRNA per cell. It is feasible to insert a drug-sensitive repressor element in front of the lentiviral/retroviral or CMV promoter in order to control their transcription rate and timing. The repressor element can be inhibited by a chemical drug or antibiotics selected from the group of G418, tetracycline, neomycin, ampicillin, kanamycin, etc, and a combination thereof.

The desired RNA molecule can be either homologous or complementary, or both, to a targeted RNA transcript or a part of the RNA transcript of a gene selected from the group consisted of fluorescent protein gene, luciferase gene, lac-Z gene, microRNA gene, miRNA precursor, transposon, jumping gene, viral gene, bacterial gene, insect gene, plant gene, animal gene, human genes, protein-coding as well as non-protein-coding genes, and their homologues, and a combination thereof. The complementary and/or homologous region of the desired RNA molecule is sized from about 12 to about 2,000 nucleotide bases, most preferably in between about 18 to about 27 nucleotide bases. The desired RNA molecule may also contain the combination of homologous and complementary sequences to an RNA transcript or a part of the RNA transcript, such as a palindromic sequence capable of forming secondary hairpin-like structures. The homology and/or complementarity rate is ranged from about 30˜100%, more preferably 35˜49% for a desired hairpin-RNA conformation and 90˜100% for both desired sense- and antisense-RNA molecules.

The present invention provides a novel means of producing aberrant RNA molecules in cell as well as in vivo, including dsRNA, siRNA, miRNA, tncRNA and shRNA compositions in vivo to induce RNAi/PTGS-associated gene silencing phenomena. Hence, the present invention provides a novel intronic RNA transcription, splicing and processing method for producing long or short sense, antisense, or both in haipin-like conformation, RNA molecules with pre-determined length and specificity. The desired intronic RNA molecule after intracellular splicing and processing can be produced in single unit or in multiple units on the recombinant gene transcript of the present invention. Same or different spliced RNA products can be generated in either sense or antisense orientation, or both, complementary to the mRNA transcript(s) of a target gene. In certain case, spliced RNA molecules complementary to a gene transcript (i.e. mRNA) can be hybridized through intracellular formation of double-stranded RNA (dsRNA) for triggering either RNAi-related phenomena with short siRNA (≦25 bp) or interferon-induced cytotoxicity with long (>25 bp) dsRNA. In other case, any small-interfering RNA (siRNA), microRNA (miRNA) and short-hairpin RNA (shRNA) molecules, or a combination thereof, can be produced as small spliced RNA molecules for inducing the RNAi/PTGS-associated gene silencing effect. The siRNA, miRNA and shRNA so obtained can be constantly produced by an expression-competent vector in vivo, thus, alleviate concerns of fast small RNA degradation. The RNA splicing-processed molecule obtained from cell culture can also be isolated and purified in vitro for generating either dsRNA or deoxyribonucleotidylated RNA (D-RNA) that is capable of triggering RNAi and/or PTGS phenomena when the molecule is transfected into a cell or an organism.

Alternatively, the present invention further provides a novel means for producing antisense microRNA (miRNA*) directed against a targeted microRNA (miRNA) in eukaryotes, resulting in inhibition of the miRNA function. Because the miRNA functions RNAi-associated gene silencing, the miRNA* can neutralize this gene silencing effect and thus rescue the function of the miRNA-suppressed gene(s). Unlike perfectly matched siRNA, the binding of miRNA* to miRNA creates a mismatched base-paired region for miRNA cleavage and degradation. Such a mismatched base-paired region is preferably located either in the middle of the stem-arm region or in the stem-loop structure of the miRNA precursor (pre-miRNA). It has been shown that mismatched base-pairing in the middle of siRNA inhibits the gene silencing effect of the siRNA (Holen et. al. (2002) Nucleic Acid Res. 30: 1757-1766; Krol et. al. (2004) J. Biol. Chem. 279: 42230-42239). Probably similar to intron-mediated enhancement (IME) phenomena in plants, previous studies in Arabidopsis and Nicotiana spp. have indicated that intronic inserts play an important role in posttranscriptional gene modulation (Rose, A. B. (2002) RNA 8: 1444-1451). The IME mechanism can recover targeted gene expression from 2 fold to over 10 fold by targeting the miRNA for silencing, which is complementary to the targeted gene.

### BRIEF DESCRIPTION OF THE DRAWINGS

The FIG. 1 depicts the biogenesis of native intronic microRNA (miRNA) that is co-transcribed with precursor messenger RNA (pre-mRNA) by Pol-II and cleaved out of the pre-mRNA by RNA splicing, while the ligated exons become a mature messenger RNA (mRNA) for protein synthesis. The spliced intronic miRNA with an antisense or a hairpin-like secondary structure is further processed into mature miRNA capable of triggering RNAi-related gene silencing effects. Thus, we designed an artificial intron incorporated in a pre-mRNA structure, namely SpRNAi, mimicking the biogenesis of the native intronic miRNA for triggering specific gene silencing via intracellular RNA splicing and processing mechanisms (FIG. 2).

The FIG. 3 depicts a novel strategy for producing desired RNA construct molecules in cells after RNA splicing and processing events occur. The oligonucleotide template of the desired RNA molecule is flanked with a RNA splicing donor and acceptor site as the same as occurs in a natural intron. The template is inserted into a gene, which is expressed by type-II RNA polymerase (Pol-II) transcription machinery under the control of either Pol-II or viral RNA promoter. In plants, type-IV RNA polymerases (Pol-IV) can also be used for such gene transcription. After transcription, the gene transcript so produced is subjected to RNA splicing and/or processing events and therefore releases the pre-designed, desired RNA molecule in the transfected cell. In certain case, the desired RNA molecule is an antisense RNA construct that can be served as antisense oligonucleotide probes for antisense gene therapy (FIG. 4). In other case, the desired RNA molecule can be of either sense or antisense orientation and possesses all element/motif/domain sequences needed for polypeptide translation and termination (FIG. 5). The polypeptide or protein encoded by the desired RNA molecule will be useful in gene replacement therapy. In some other cases, the desired RNA molecule consists of small antisense and sense RNA fragments to function as double-stranded siRNA for RNAi induction (FIG. 5). In yet other cases, the desired RNA molecule is a hairpin-like RNA construct capable of causing RNAi-associated gene silencing phenomena (FIG. 6).

Referring particularly to the drawings for the purpose of illustration only and not limitation, there is illustrated:

FIG. 1 depicts the intracellular mechanism of natural microRNA (miRNA) biogenesis from gene intron.

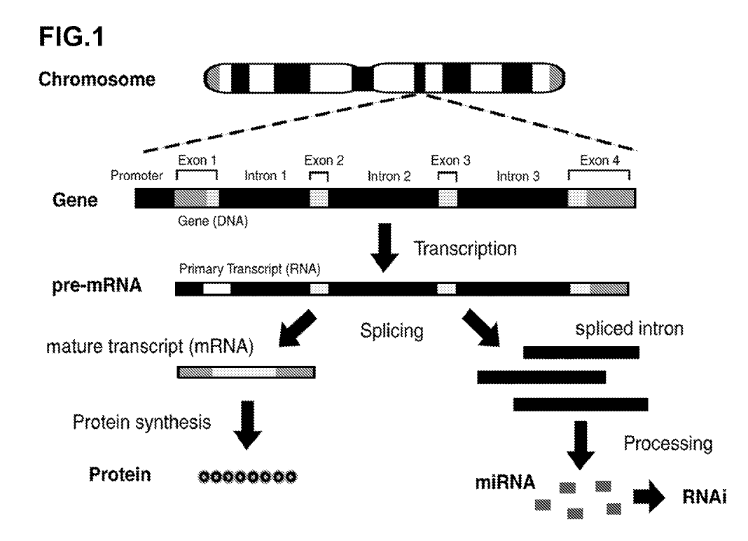

FIG. 2 depicts the strategy of using intron to generate man-made RNAi molecule (e.g. miRNA), namely SpRNAi, mimicking the biogenesis of a natural intronic miRNA.

FIG. 3 depicts the principal embodiment of the SpRNAi-containing recombinant gene (SpRNAi-rGFP) construct, construction, and the relative applications thereof.

FIG. 4 depicts the first preferred embodiment of antisense RNA generation by spliceosome cleavage from retroviral (e.g. LTR) promoter-mediated precursor transcripts.

FIG. 5 depicts the second preferred embodiment of sense and antisense siRNA generation by spliceosome cleavage from viral (e.g. CMV or AMV) promoter-mediated precursor transcripts.

FIG. 6 depicts the third preferred embodiments of hairpin-like RNA generation by spliceosome cleavage from Pol-II (e.g. TRE or Tet-On/Off responsive element) promoter-mediated precursor transcripts.

FIG. 7 shows the microscopic results of Example 5, showing interference of green fluorescent protein (eGFP) expression in rat neuronal stem cells by various SpRNAi-rGFP constructs made from Examples 2 and 3.

FIG. 8 shows the Western blotting results of Examples 5 and 6, showing interference of green fluorescent protein (eGFP) expression in rat neuronal stem cells by various SpRNAi-rGFP constructs made from Examples 2 and 3.

FIG. 9 shows the Western blotting results of Examples 5 and 6, showing interference of integrin β1 (ITGb1) expression in human prostatic cancer LNCaP cells by various SpRNAi constructs made from Example 3.

FIGS. 10A-B show the Northern blot analysis of SpRNAi-induced cellular gene silencing against HIV-1 infection as described in Example 7.

FIG. 11 depicts the different mechanisms between conventional siRNA-mediated RNAi and present SpRNAi-mediated gene silencing phenomena.

FIGS. 12A-C show different designs of intronic RNA inserts in an SpRNAi construct for microRNA biogenesis and the resulting gene silencing of targeted green fluorescent protein (EGFP) expression in zebrafish, demonstrating an asymmetric design preference in transfection of (1) 5′-miRNA*-stemloop-miRNA-3′ and (2) 5′-miRNA-stemloop-miRNA*-3′ hairpin RNA structures, respectively, as described in Example 9.

FIGS. 13A-B show the generation of transgenic loss-of-FMRP-gene-function zebrafish in vivo, using the present invention for disease model research as described in Example 10. Because the Tg(UAS:gfp) zebrafish expresses green GFP and the anti-FMRP SpRNAi transgene is marked with red GFP, we can easily observe the normal dendritic neurons (green) versus the loss-of-FMRP-function neurons (yellow) in fish brain.

FIGS. 14A-D show the gene silencing effects of the Example 11 on organ development in transgene-like chicken embryos, including liver and skin with β-catenin gene knockout (A)-(C) and beak with noggin gene knockdown (D) in vivo.

FIG. 15 shows the gene silencing effect of the Example 12 on skin pigmentation in mouse in vivo, indicating localized transgenic gene modulation controlled by the use of the present invention.

FIGS. 16A-B show the Northern blot analysis of HIV gene silencing using the present invention as described in Example 13. Complete suppression of HIV-1 replication using SpRNAi transfection against gag/pro/pol viral genes in patients' CD4⁺ T lymphocytes was observed, indicating a feasible vaccination strategy for AIDS therapy and treatment of viral infection.

FIG. 17 shows the inhibitory effect of an antisense microRNA (miRNA*) on its targeted microRNA (miRNA) function, resulting in cancellation of the miRNA-mediated gene silencing effect and recovery of the miRNA-targeted gene expression, such as integrin β1 (ITGb1) described in the Example 14.

### DETAILED DESCRIPTION OF THE INVENTION

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

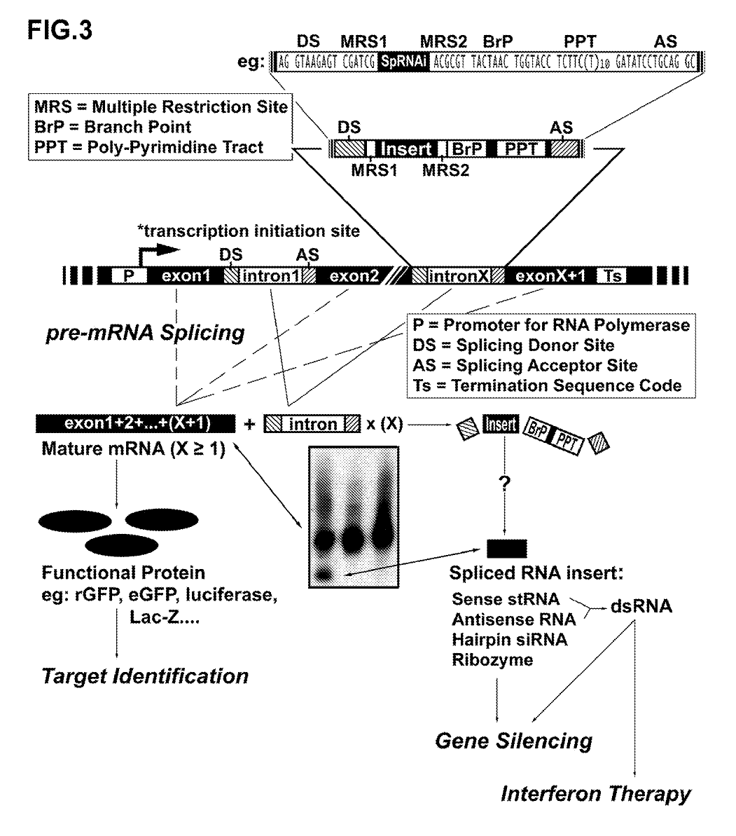

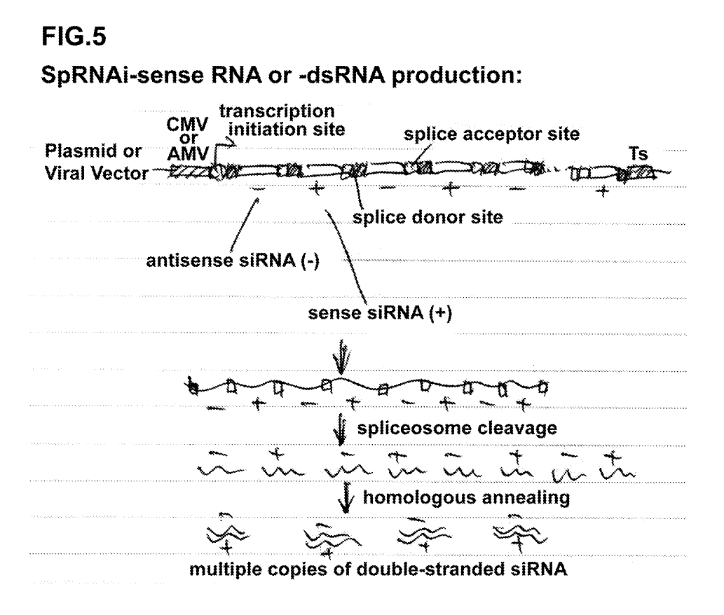

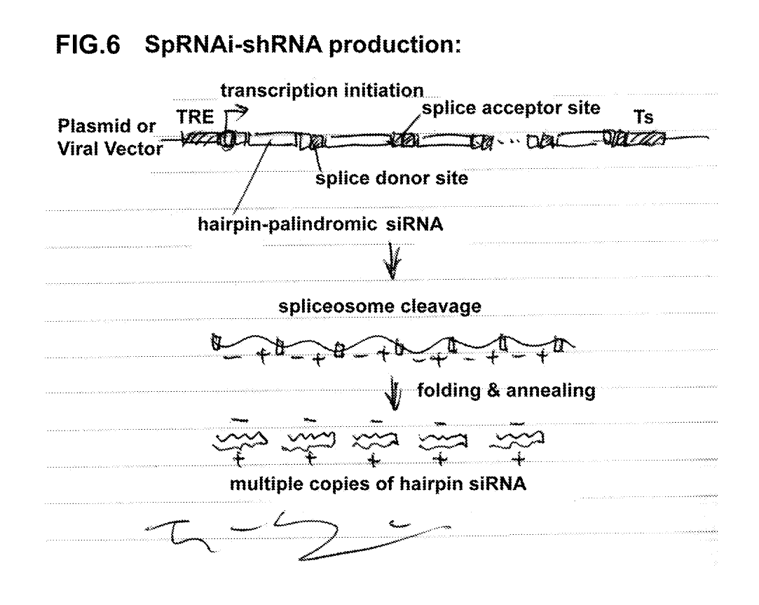

The present invention provides a novel composition and method for altering genetic characteristics of a cell or an organism. Without being bound by any particular theory, such an alteration of genetic characteristics is directed to a newly discovered intron-mediated gene silencing mechanism, triggered by transfection of an artificially recombinant gene containing at least an RNA splicing- and processing-competent intron (SpRNAi) construct in the cell or organism of interest. The SpRNAi intron carries an intronic RNA insert, which can be released by intracellular RNA splicing and processing machineries and then triggers RNAi and/or PTGS-related gene silencing effects on complementary gene targets. Generally, as shown in FIGS. 3 and 4-6, when the recombinant gene is chemically transduced, liposomally transfected, or otherwise introduced by viral infection into the target cell or organism, small intronic RNA fragments of the SpRNAi inserts are produced and then released from the recombinant gene transcript by RNA splicing and/or processing machineries, such as spliceosome and P body. These intronic RNA fragments can form lariat-form RNA, short-temporary RNA (stRNA), antisense RNA, small-interfering RNA (siRNA), double-stranded RNA (dsRNA), short-hairpin RNA (shRNA), microRNA (miRNA), tiny non-coding RNA (tncRNA), aberrant RNA which may contains mismatched base pairing, deoxynucleotidylated RNA (D-RNA), ribozyme RNA, or a combination thereof, and is therefore able to induce RNA interference (RNAi) and/or posttranscriptional gene silencing (PTGS) effects on targeted gene expression. Consequently, the targeted gene transcripts (i.e. mRNA) are either degraded by RNA-dependent endonucleases (RDE), such as RNaseIII endonucleases (Dicer), or suppressed translationally by RNA-induced silencing complex (RISC) and/or RNAi-induced initiator of transcriptional silencing (RITS).

Similar to natural mRNA splicing and processing processes, the spliceosome machinery catalyzing intron removal in our design of the present invention is formed by sequential assembly of intracellular spliceosomal components on several snRNP-recognition elements of the SpRNAi intron (e.g. binding sites for snRNPs U1, U2 and U4/U6.U5 tri-snRNP). The methods for incorporating synthetic snRNP-recognition elements in an SpRNAi intron are described in Examples 2 and 3. In brief, a sequential assembly of the snRNPs to their recognition elements has been proposed: first, binding of U1 snRNP to the 5′-splicing junction (splicing donor site), then binding of U2 snRNP to a branch-point site, and last, association of the U4/U6.U5 tri-snRNP to the U1 and U2 snRNPs, so as to form an early splicing complex for precisely cleavage of the 5′-splicing junction. On the other hand, the 3′-splicing junction (splicing acceptor site) is cleaved by a late splicing complex formed by U5 snRNP and some other splicing proteins after the release of the 5′-splicing junction. However, little is known about the protein/protein and RNA/protein interactions that bridge the U4/U6 and U5 snRNP components within a eukaryotic tri-snRNP, and knowledge on the binding sites of proteins on U4/U6 and U5 snRNPs remains largely unclear.

Design of Artificially Recombinant Genes for Testing Intron-Mediated Gene Silencing Effects

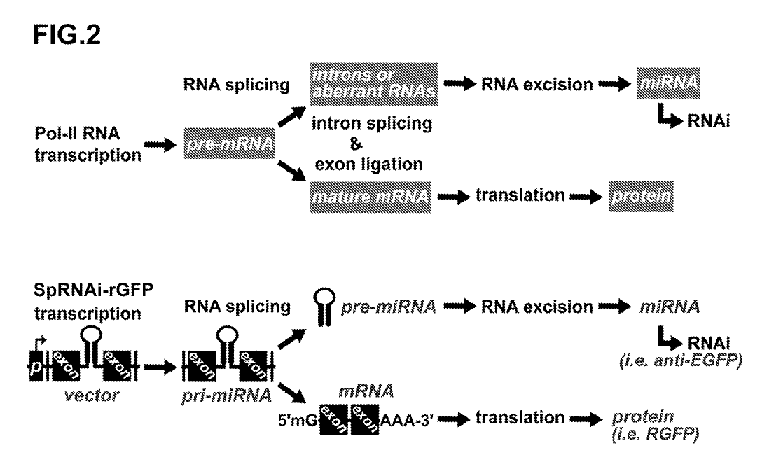

Strategy for molecular analysis of intracellular RNA splicing- and processing-directed gene silencing mechanisms was tested using an artificial recombinant gene, namely SpRNAi-rGFP (FIGS. 2 and 3). Recombination of a splicing-competent intron (SpRNAi) in a red fluorescent protein gene (rGFP) was genetically engineered by sequential ligation of synthetic DNA sequences as shown in Examples 1-3. The SpRNAi intron further comprises an intronic insert, which can be released by intracellular RNA splicing and/or processing machineries, then triggering an intron-mediated gene silencing mechanism through the transcription and splicing of the SpRNAi-rGFP gene. Although we showed here a model of intron-mediated gene silencing functioning via Pol-II pre-mRNA splicing, the same principle can be used to design intronic inserts functioning via the RNA processing of pre-ribosomal RNA (pre-rRNA), which is mainly transcribed by type-I RNA polymerases (Pol-I). In plants, both Pol-II and Pol-IV can function as an RNA-dependent RNA polymerase (RdRp) for generating intronic RNA inserts. Other RNA transcripts capable of being used to express and process the intronic RNA inserts include mRNA, hnRNA, rRNA, TRNA, snoRNA, snRNA, microRNA, viral RNA, and their precursors as well as derivatives.

The SpRNAi intron is flanked with a splicing donor (DS) and acceptor (AS) site, and contains at least one anti-gene insert, a branch point (BrP) and a poly-pyrimidine tract (PPT) in between the DS-AS sites for interacting with intracellular spliceosomes. Using low stringent Northern blotting analysis (middle bottom of FIG. 3), we were able to observe the release of 15˜45-nucleotide intronic RNA fragments from the designed SpRNAi-rGFP gene transcript (left), but neither from an intron-free rGFP (middle) nor from a defective SpRNAi-rGFP (right) RNA without a functional splicing donor site, while spliced exons were linked to form mature RNA for reporter rGFP protein synthesis. As shown in Examples 5-14, we have successfully tested short sense, antisense and hairpin constructs of many anti-gene intronic inserts for triggering targeted gene silencing in human prostate cancer LNCaP, human cervical cancer HeLa and rat neuronal stem HCN-A94-2 cells as well as in zebrafish (vertebrate), chicken (avian) and mouse (mammal) in vivo.

As shown in FIG. 3, splicing-competent introns (SpRNAi) were synthesized and inserted into an intron-free red fluorescent protein gene (rGFP; RGFP) that was mutated from the HcRed1 chromoproteins of Heteractis crispa. Because the inserted SpRNAi intron(s) disrupted the functional fluorescent protein structure of rGFP, we were able to check the intron removal and mRNA maturation of rGFP gene transcripts through the reappearance of red fluorescent light emission at the 570-nm wavelength in a successfully transfected cell or organism. Construction of SpRNAi was based on the natural structures of a precursor messenger RNA (pre-mRNA) intron, consisting of spliceosomal recognition components, such as splicing donor and acceptor sites in both ends, respectively, for precise cleavage, a branch point domain for splicing recognition, a poly-pyrimidine tract for spliceosomal interaction, linkers for connection of each major recognition components and some restriction/cloning sites for desired intronic insertion.

The splicing donor site is an oligonucleotide motif containing homology to (5′-exon-AG)-(splicing point)-GTA(A/-)GAG(G/T)-3′ sequences (SEQ.ID.NO.1), including but not limited, 5′-AG GTAAGAGGAT-3′, 5′-AG GTAAGAGT-3′, 5′-AG GTAGAGT-3′, 5′-AG GTAAGT-3′, etc. The splicing acceptor site is another oligonucleotide motif comprising homology to 5′-G(W/-)(T/G)(C/G)C(T/C)(G/A)CAG-(splicing point)-(G/C-3′-exon) sequences (while W is a pyrimidine A, T or U) (SEQ.ID.NO.2), including but not limited, 5′-GATATCCTGCAG G-3′, 5′-GGCTGCAG G-3′, 5′-CCACAG C-3′, etc. The branch point is an “A” nucleotide located within an oligonucleotide element/domain homologous to 5′-TACT(A/T)A*(C/T)(-/C)-3′ sequences (while the symbol “*” marks the branch site) (SEQ.ID.NO.3), including but not limited, 5′-TACTAAC-3′, 5′-TACTTATC-3′ and so on. The poly-pyrimidine tract is a high T or C content oligonucleotide sequence homologous to 5′-(TY)m(C/-)(T)nC(C/-)-3′ or 5′-(TC)nNCTAG(G/-)-3′ (while Y is a C or T/U and the “-” means an empty site). The symbols of “m” and “n” indicates multiple repeats ≧1; most preferably, m=1˜3 and n=7˜12. For all of the above spliceosomal recognition components, the deoxythymidine (T) nucleotide is replaceable with a uridine (U).

To test the function of a spliced intron, various oligonucleotide inserts were cloned into the SpRNAi through restriction/cloning sites, respectively. The restriction/cloning site is preferably generated by a restriction enzyme selected from the group of AatII, AccI, AflII/III, AgeI, ApaI/LI, AseI, Asp718I, BamHI, BbeI, BclI/II, BglII, BsmI, Bsp120I, BspHI/LU11I/120I, BsrI/BI/GI, BssHII/SI, BstBI/UI/XI, ClaI, Csp6I, DpnI, DraI/II, EagI, Ecl136II, EcoRI/RII/47III, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu 101, PstI, PvuI/II, RsaI, SacI/II, SalI, Sau3AI, SmaI, SnaBI, SphI, SspI, StuI, TaiI, TaqI, XbaI, XhoI, XmaI endonuclease, and a combination thereof. These intron inserts are DNA templates encoding aberrant RNAs selected from the group consisting of lariat-form RNA, short-temporary RNA (stRNA), antisense RNA, small-interfering RNA (siRNA), double-stranded RNA (dsRNA), short-hairpin RNA (shRNA), microRNA (miRNA), aberrant RNA containing mismatched base pairing, long deoxynucleotidylated RNA (D-RNA), ribozyme RNA and their precursors as well as derivatives in either sense or antisense orientation, or both, and a combination thereof.

As shown in Example 5, the expression of a hairpin-like RNA insert from SpRNAi-rGFP often induces a much stronger gene silencing effect than those of sense and antisense RNA inserts, showing an average of >80% knockdown specificity to all targeted gene transcripts. This knockdown specificity is determined by both of the complementarity and homology between the intronic insert and its targeted gene transcript. For example, the most effective hairpin-SpRNAi insert often possessed about 40˜42% homology and 40˜42% complementarity to the targeted gene domain, while an A/T-rich linker sequence filled in the rest 6˜20% space as a loop conformation. To those sense- and antisense-SpRNAi inserts, although the homology or complementarity can be increased up to 100% in one orientation, an average of 40˜50% knockdown efficacy was observed. Therefore, depending on the homology and complementarity between the intronic insert and the targeted gene transcript, we can design and use different kinds of intronic inserts, either respectively or together, to manipulate specific gene expression at a desired level in the cell or organism of interest.

Simultaneous Expression of rGFP and Silencing of eGFP by SpRNAi Transfection

For the convenience of gene delivery and activation in tested cells or organisms, an SpRNAi-containing recombinant gene is preferably cloned into an expression-competent vector, selected from the group consisting of plasmid, cosmid, phagmid, yeast artificial chromosome, transposon, jumping gene, viral vector, and the combination thereof. The vector is introduced into the cell, tissue, plant or animal organism by a high efficient gene delivery method selected from the group consisting of liposomal transfection, chemical transfection, chemical transformation, electroporation, transposon recombination, jumping gene insertion, viral vector infection, micro-injection, gene-gun penetration, and a combination thereof.

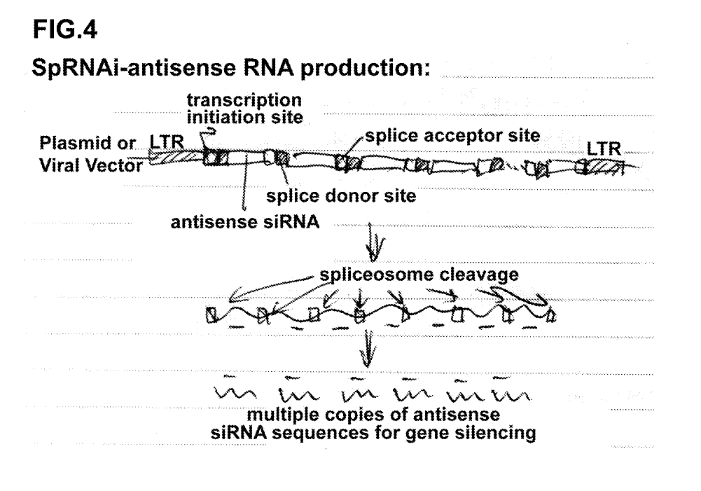

As shown in FIGS. 4-6, the vectors contain at least one viral or type-II RNA polymerase (Pol-II) promoter, or both, for expressing the SpRNAi-containing gene in eukaryotic cells, a Kozak consensus translation initiation site to increase translation efficiency in eukaryotic cells, multiple SV40 polyadenylation signals downstream of the SpRNAi-containing gene for processing the 3′-end of the recombinant gene transcript, a pUC origin of replication for propagation in prokaryotic cells, at least two restriction/cloning sites for cloning the SpRNAi-containing gene, an optional SV40 origin for replication in mammalian cells that express the SV40 T antigen and an optional SV40 early promoter for expressing antibiotic resistance gene in replication-competent prokaryotic cells. The expression of antibiotic resistance genes is used to serve as a selective marker for searching of successfully transfected or infected clones, possessing resistance to the antibiotics selected from the group consisted of G418, penicillin G, ampcillin, neomycin, paromycin, kanamycin, streptomycin, erythromycin, spectromycin, phophomycin, tetracycline, rifapicin, amphotericin B, gentamicin, chloramphenicol, cephalothin, tylosin, and a combination thereof.

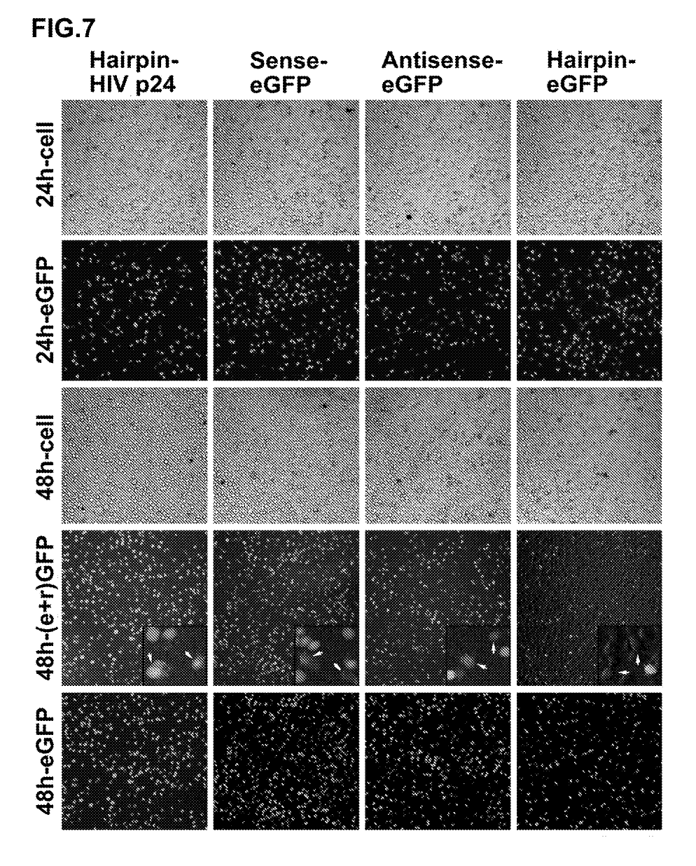

As shown in FIG. 7 and Example 5, transfection of the plasmid vectors containing various SpRNAi-rGFP recombinant genes directed against an enhanced green fluorescent protein (eGFP) was found to be successful in both expression of rGFP (red) and silencing eGFP (green). The use of eGFP-positive rat neuronal stem cell clones provided an excellent visual aid to measure the silencing effects of various SpRNAi inserts. Rat neuronal stem cell clones AP31 and PZ5a were primary cultured and maintained as described in Example 1. Observing from the cell culture after 24-hr transfection, almost the same amount of total cell number and eGFP-positive cell population were well seeded and very limited apoptotic or differentiated cells occurred. Silencing of eGFP emission was detected at the 518-nm wavelength 36˜48 hours (hr) after transfection, indicating a potential onset timing required for the release of small interfering inserts from the SpRNAi-rGFP gene transcripts by spliceosomal and RISC machineries. Because all successfully transfected cells displayed red fluorescent emission at about 570-nm wavelength, we were able to trace the gene silencing effect by measuring relative light intensity of the green fluorescent emission in the red fluorescent cells, showing a knockdown potency of hairpin-eGFP>>sense-eGFP≈antisense-eGFP>>hairpin-HIV p24 (negative control) inserts.

Western Blot Analysis of RNA Splicing- and Processing-Directed eGFP Silencing Effects

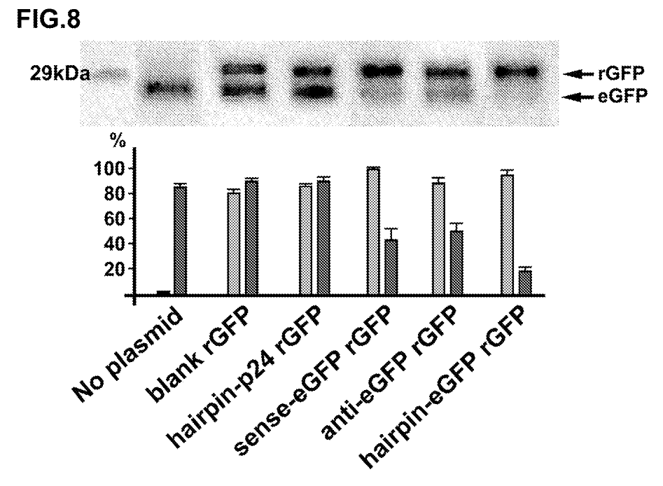

As shown in FIG. 8, quantitative knockdown levels of eGFP protein in the rat neuronal stem cell clones AP31 and PZ5a by various SpRNAi inserts were measured on an unreduced 6% SDS-polyacrylamide gel. For normalizing the loading amounts of transfected cellular proteins, rGFP protein levels (˜30 kDa, red bars) were adjusted to be comparatively equal, representing an average expression range from 82 to 100% intensity (Y axis). The eGFP levels (27 kDa; green bars) were reduced by the transfection of SpRNAi-rGFP genes containing sense-eGFP (43.6%), antisense-eGFP (49.8%) and hairpin-eGFP (19.0%) inserts, but not that of intron-free rGFP gene (blank control) or SpRNAi-rGFP gene containing hairpin-HIV p24 insert (negative control). These findings confirm the above knockdown potency of hairpin-eGFP>>sense-eGFP≈antisense-eGFP>>hairpin-HIV p24 (negative control), and also demonstrate that only an anti-gene insert with either high homology or high complementarity, or both, to the targeted gene transcript can elicit this intron-mediated gene silencing effect.

Western Blot Analysis of RNA Splicing- and Processing-Directed Integrin β1 Silencing in Human Prostatic Cancer Cells

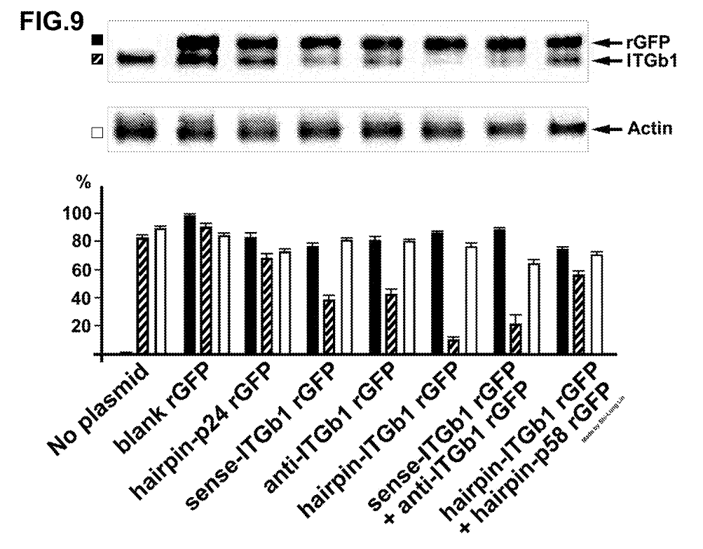

As shown in FIG. 9, a similar RNA splicing- and processing-directed gene silencing phenomenon was observed in human cancerous LNCaP cells. Quantitative knockdown levels of integrin β1 (ITGb1) protein by various intronic SpRNAi inserts were measured on a reduced 8% SDS-polyacrylamide gel. The relative amounts of rGFP (black bars), ITGb1 (gray bars) and actin (white bars) were shown by a percentage scale (Y axis). The ITGb1 levels (29 kDa) were significantly reduced by the transfection of SpRNAi-rGFP genes containing sense-ITGb1 (37.3%), antisense-ITGb1 (48.1%) and hairpin-ITGb1 (13.5%) inserts, but not that of intron-free rGFP gene (blank control) or SpRNAi-rGFP gene containing hairpin-HIV p24 insert (negative control). Co-transfection of SpRNAi-rGFP genes containing sense- and antisense-ITGb1 inserts elicited a significant gene silencing effect (22.5%) in company with 10˜15% cell death, while that of SpRNAi-rGFP genes containing hairpin-ITGb1 and hairpin-p58/HHR23 inserts partially blocked the splicing-directed gene silencing effect to achieve an average 57.8% expression level. These findings indicate that the SpRNAi-induced gene silencing effect may function in a wide range of genes and cell types of interest.

Strategy for HIV Vaccination Using SpRNAi-rGFP Vector Transfection

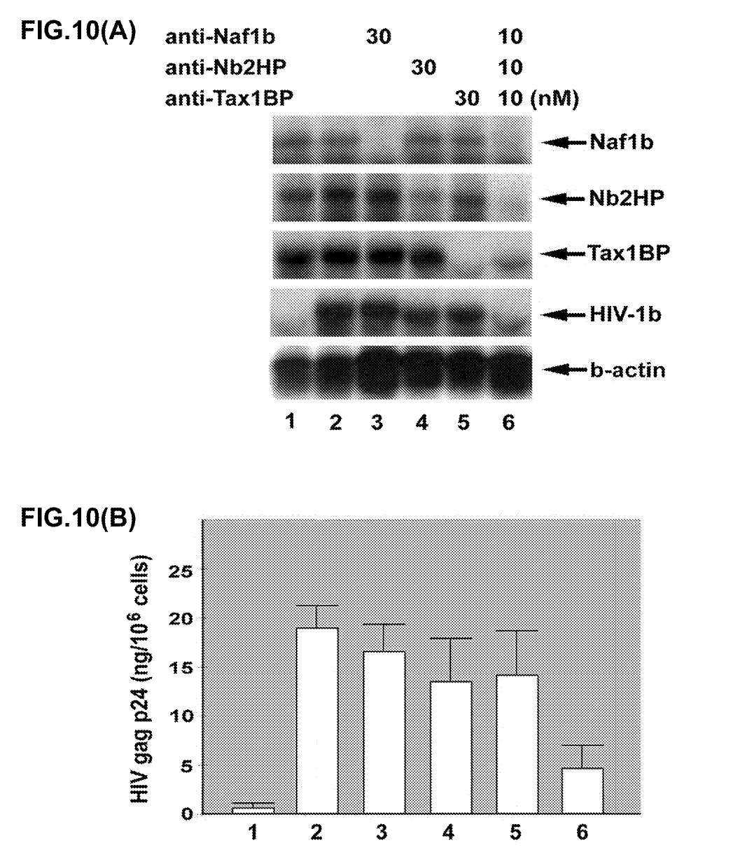

Northern blot analysis of SpRNAi-mediated gene silencing directed against HIV-promoted cellular genes is shown to inhibit HIV infection (FIGS. 10A-B). Feasibility of AIDS vaccination was tested using SpRNAi-derived intronic miRNA directed against cellular genes as anti-HIV drugs. FIG. 10A, the Northern blot result of SpRNAi-induced gene silencing effects on Naf1β, Nb2HP and Tax1BP was shown to prevent HIV-1 infection. The tested gene targets were selected through RNA-PCR microarray analysis of differential expression genes from the acute (one˜two week) and chronic (about two year) infected patients' primary T cells with or without 25 nM anti-HIV D-RNAi treatment (Lin et. al. (2001) supra). The SpRNAi-recombinant gene vector concentrations of all treatments were normalized to 30 nM in total. FIG. 10B displays the bar chart of HIV gag-p24 ELISA results (white) in correlation to the treatment results of FIG. 10A.

Because CD4 has normal function in IL-2 stimulation and T-cell growth activation, the CD4 receptor may not be an ideal target for HIV prevention. However, the search for HIV-dependent cellular genes in vivo was hindered by the fact that infectivity of viruses and infection rate among different patients are usually different leading to inconsistent results. Short-term ex-vivo culture conditions can normalize infectivity and infection rate of HIV transmission in a more uniform CD4⁺ T cell population. Microarray analysis based on such ex vivo conditions would be reliable for critical biomedical and genetic research of HIV-1 infection. Our studies of microarray-identified differential gene profiles between HIV⁻ and HIV⁺ T cells in the acute and chronic infection phases has provided many potential anti-HIV cellular gene targets for AIDS therapy and prevention. To functionally evaluate the usefulness of targeting cellular genes for HIV vaccination, three highly differentially expressed genes, Naf1β, Nb2 homologous protein to Wnt-6 (Nb2HP) and Tax1 binding protein (Tax1BP), has been tested to inhibit HIV-1 infectivity.

Because each of them contributes only parts of AIDS complications, knockdown of single target gene failed to suppress HIV-1 infection, while combination of all three SpRNAi drugs at the same total concentration showed a significant 80±10% reduction of HIV-1 infection (p<0.01). As shown in FIG. 10A, Northern blot results were shown from left to right: (lane 1) normal T cells without HIV infection (blank controls); (lane 2) HIV-infected T cells (positive controls); (lane 3) anti-Naf1β/SpRNAi treatment of (2); (lane 4) anti-Nb2HP SpRNAi treatment of (2); (lane 5) anti-Tax1BP SpRNAi treatment of (2); and (lane 6) combined treatment of (3), (4) and (5). In the same experiment, the ELISA results of HIV gag-p24 protein (FIG. 10B) also correlated with the Northern blot data, showing an average of 77±5% reduction of gag-p24 expression. These findings suggest that the intron-mediated gene modulation is capable of repelling viral infection through concurrently multiple gene silencing and therefore point to a useful strategy for the development of viral vaccination and therapy.

Different Mechanisms Between siRNA-Mediated and SpRNAi-Mediated RNAi

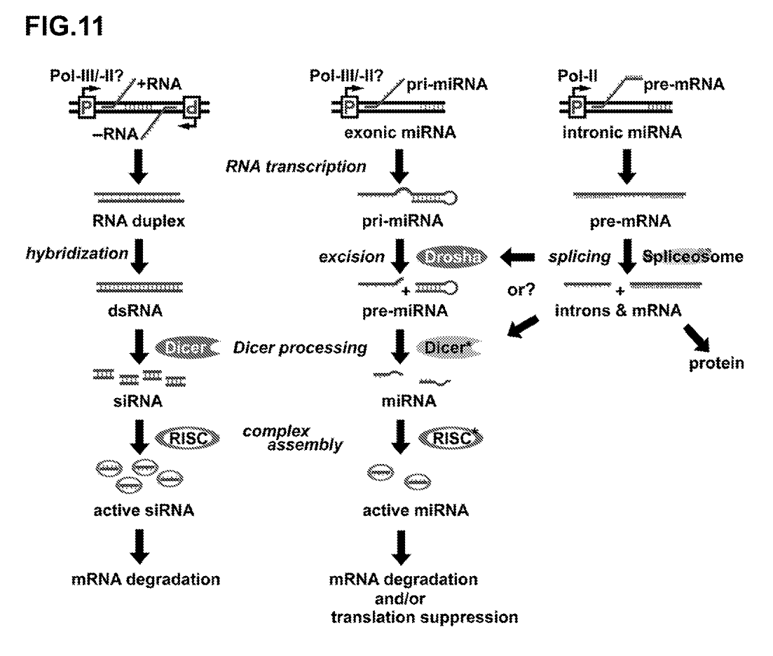

Although an in vitro model of siRNA-mediated RNAi has been proposed, the characteristics of Dicer and RNA-induced silencing complex (RISC) are distinctly different in the siRNA and miRNA mechanisms (Tang, G. (2005) Trends Biochem Sci. 30: 106-114). FIG. 11 shows the comparison of biogenesis and RNAi mechanisms among siRNA and intronic microRNA (Lin et. al. (2005) Gene 356: 32-38). SiRNA is formed by hybridization of two perfectly complementary RNAs and further processed into 19-22 bp RNA duplexes by RNaseIII-familial endonucleases, namely Dicer; while miRNA biogenesis involves five steps: First, a long primary precursor miRNA (pri-miRNA) is transcribed by RNA polymerases type II (Pol-II). Second, the long pri-miRNA is excised by Drosha-like RNaseII endonucleases and/or spliceosomal components, depending on the origin of the pri-miRNA located in an exon or an intron, respectively, to form precursor miRNA (pre-miRNA), and third, the pre-miRNA is exported out of the nucleus by Ran-GTP and the receptor Exportin-5. In the cytoplasm, Dicer-like nucleases cleave the pre-miRNA to form mature miRNA. Lastly, the mature miRNA and siRNA are incorporated into a ribonuclear protein particle (RNP), respectively, and forms RISC assembly containing either strand of the siRNA or the single-stranded miRNA. The RISC is capable of executing RNAi-related gene silencing. The RISC action of miRNA is however considered to be more specific and less adverse than that of siRNA because only one strand conformation is involved. SiRNA primarily triggers mRNA degradation, whereas miRNA can induce either mRNA degradation or suppression of protein synthesis, depending on the sequence complementarity to its targeted gene transcripts.

Intron-Mediated Gene Silencing in Zebrafish In Vivo

The foregoing discussion establishes the fact that intronic miRNAs are effective strategy for silencing targeted gene expression in vivo. We first tried to determine the structural design of pre-miRNA inserts for the best gene silencing effect. We found that a strong structural preference presents in the selection of mature miRNA for assembly of the RNAi effector, RNA-induced gene silencing complex (RISC). RISC is a protein-RNA complex that directs either targeted gene transcript degradation or translational repression through the RNAi mechanism.

In zebrafish, we have observed that the stem-loop structure of pre-miRNA determines the sequence of mature miRNA for RISC assembly, which is different from siRNA-associated RISC assembly (Lin et. al. (2005) Gene 356: 32-38). Formation of siRNA duplexes plays a key role in assembly of the siRNA-associated RISC. The two strands of the siRNA duplex are functionally asymmetric, but assembly into the RISC complex is usually preferential for only one strand. Such a preference is determined by the thermodynamic stability of each 5′-end base-pairing in the siRNA strand. Based on this siRNA model, the formation of miRNA and its complementary miRNA (miRNA*) duplex was thought to be an essential step for the assembly of miRNA-associated RISC. If this were true, no functional bias would be observed in the stem-loop structure of pre-miRNA. However, we observed that the stem-loop of the intronic pre-miRNA was involved in the strand selection of a mature miRNA for RISC assembly.

In experiments, we constructed miRNA-expressing SpRNAi-RGFP vectors as described in Example 3 and two symmetric pre-miRNAs, miRNA-stemloop-miRNA* (1) and miRNA*-stemloop-miRNA (2), were synthesized and inserted into the vectors, respectively. Both pre-miRNAs contained the same double-stranded stem-arm region, which was directed against the EGFP nt 280-302 sequence. Because the intronic insert region of the SpRNAi-RGFP recombined gene is flanked with a PvuI and an MluI restriction site at the 5′- and 3′-ends, respectively, the primary insert can be easily removed and replaced by various anti-gene inserts (e.g. anti-EGFP) possessing cohesive ends. By allowing changes in the SpRNAi insert directed against different gene transcripts, this intronic miRNA biogenesis system provides a valuable tool for genetic and miRNA-associated research in vivo.

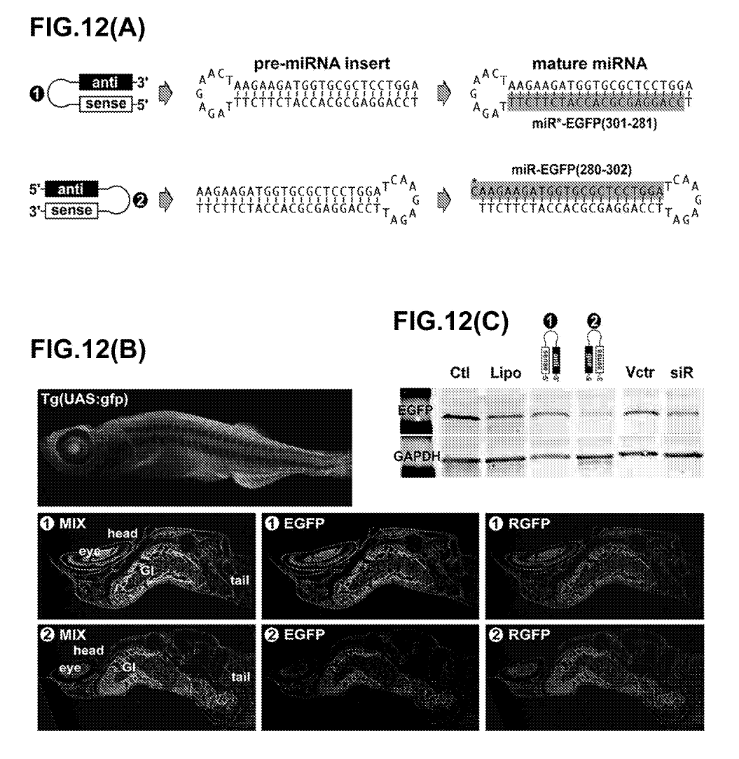

To determine the structural preference of the designed pre-miRNAs, we isolated the zebrafish small RNAs by mirVana miRNA isolation columns (Ambion, Austin, Tex.) and then precipitated all potential miRNAs complementary to the target EGFP region by latex beads containing the target RNA sequence. One full-length miRNA, miR-EGFP(280-302), was verified to be active in transfection of the 5′-miRNA-stemloop-miRNA*-3′ construct, as shown in FIG. 12A (gray-shading sequences). Because the mature miRNA was detected only in the zebrafish transfected by the 5′-miRNA-stemloop-miRNA*-3′ construct, the miRNA-associated RISC tends to preferably interact with the construct (2) rather than the (1) pre-miRNA. The green fluorescent protein EGFP expression was constitutively driven by the β-actin promoter located in almost all cell types of the zebrafish, while FIG. 12B shows that transfection of the SpRNAi-RGFP vector into the Tg(UAS:gfp) zebrafish co-expressed a red fluorescent protein RGFP, serving as a positive indicator for miRNA generation in the transfected cells. We applied the liposome-encapsulated SpRNAi-RGFP vector to the fish and found that all vectors completely penetrated the two-week-old zebrafish larvae within 24 hours, providing fully systemic delivery of the miRNA.

The indicator RGFP (red) was evenly detected in the fish transfected by either (1) or (2) pre-miRNA, whereas the silencing effect on targeted EGFP (green) was observed only in the fish transfected by the 5′-miRNA-stemloop-miRNA*-3′ (2) pre-miRNA, showing a mixed orange rather than wildtype yellow color. As shown in FIG. 12C, Western blot analysis confirmed the same gene silencing results, demonstrating a >85% RGFP knockdown in the (2)-transfected fish. The suppression level in gastrointestinal (GI) tract area was low, probably due to a high RNase activity in this region. Because the same 5′-thermostability is applied to both pre-designed pre-miRNA stem-arms, we suggest that the stem-loop structure of pre-miRNA is involved in the strand selection of mature miRNA for RISC assembly. Given that the cleavage site of Dicer in the stem-arm determines the strand selection of mature miRNA, the stem-loop of pre-miRNA may function as a determinant for the recognition of a special cleavage site. Therefore, the heterogeneity of stem-loop structures among various miRNA species may help to explain the evolution of native miRNA in vertebrates.

Generation of Novel Transgenic Animal Models In Vivo Using the Present Invention

Fragile X syndrome (FraX) is the most common form of inherited mental retardation, with the estimated prevalence of 30% of total human mental retardation disorders, and is also among the most frequent single gene disorders. The gene affected by the syndrome in 99% patients, FMR1, is transcriptionally inactivated by the expansion and the methylation of trinucleotide (CGG) repeats, located in the 5′-untranslated region (5′-UTR) of the gene. This 5UTR r(CGG) expansion region was proposed to be the native anti-FMR1 microRNA target site of human FraX disorder (Jin et al. (2004) Nat Cell Biol. 6: 1048-1053). Native anti-FMR1 miRNA triggers the formation of RNA-induced initiator of transcriptional gene silencing (RITS) on the homologous (CGG) repeats and leads to heterochromatin repression of the FMR1 locus. FMR1 encodes an RNA-binding protein, FMRP, which is associated with polyribosome assembly in an RNA-dependent manner and capable of suppressing protein translation through an RNA interference (RNAi)-like pathway that is important for neuronal development and plasticity. However, no appropriate animal model is available for the study of FraX etiology because current Drosophila and mouse models are all based on the gene deletion of FMRP, completely irrelevant to the mechanism of RNAi.

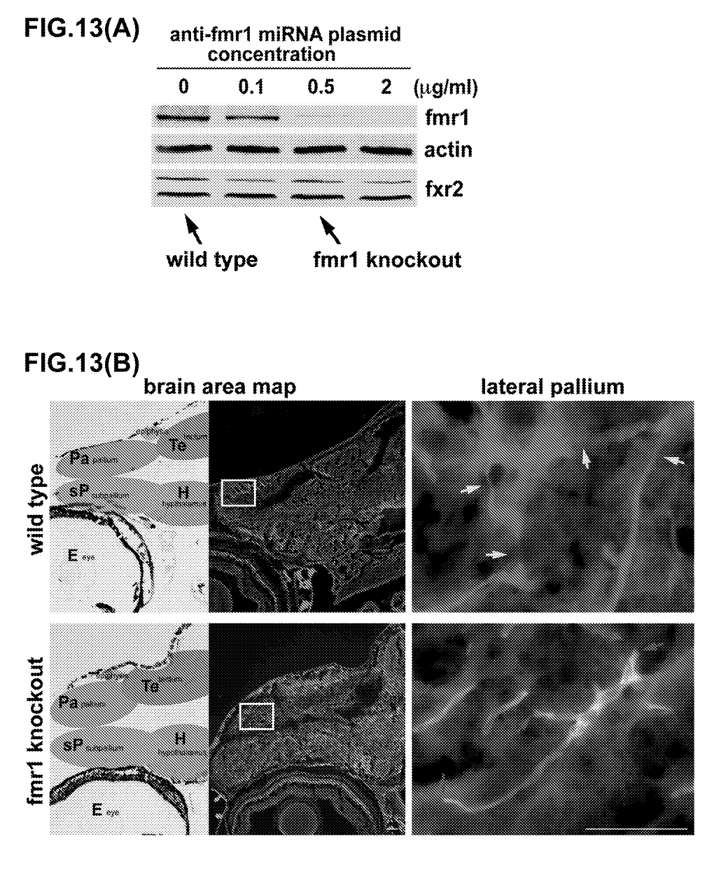

To investigate the role of microRNA (miRNA) in this proposed disease model, we have designed and tested man-made miRNA transgenes directed against the fish fmr1 gene to generate loss-of-function transgenic zebrafish. After transgenic transfection as shown in FIG. 13A, the zebrafish fmr1 levels were shown to be inversely correlated to the concentrations of the anti-fmr1 miRNA-expressing SpRNAi-rGFP plasmid as determined by Western blot analysis (Example 10). No gene silencing effect was observed in off-target and house-keeping genes, such as fxr1,2 and actin. Line chart (right) shows the inverse correlation between the fmr1 expression level and the concentration of the anti-fmr1 miRNA-expressing plasmid used in transfection. Arrows indicate the two samples which were further used in comparison as shown in FIG. 13B. After miRNA-mediated fmr1 gene silencing was confirmed, we further compared the changes of brain development between wildtype and fmr1-knockdown zebrafish (indicated by black arrows as shown in FIG. 13A). About 90% of zebrafish embryos remained viable after transfected with 0.5 μg/ml of the anti-fmr1 miRNA-expressing SpRNAi-rGFP plasmid.

As shown in FIG. 13B, fluorescent 3D-micrograph showed abnormal neuron morphology and connectivity in the loss-of-fmr1-function transgenics, similar to those in human FraX. In fish lateral pallium, wildtype neurons presented normal dendritic outline and well connection to each other (yellow arrows), whereas the transgenics exhibited thin, strip-shape neurons, reminiscent of the abnormal dendritic spine neurons in the human FraX. Altered synaptic plasticity has been reported to be a major physiological damage in the FraX of human and mouse, particularly in the hippocampal stratum radiatum area. Synapse deformity frequently occurred in the loss-of-fmr1-function neurons (red arrows), indicating the functional role of FMR1 in activity-dependent synaptic neuron plasticity. Further, the group 1 metabotropic glutamate receptor (mGluR)-activated long-term depression (LTD) could be augmented in the absence of fmr1, suggesting that exaggerated LTD may be responsible for aspects of abnormal neuronal responses in FraX, such as autism. As a result, future therapy and research based on this novel FraX model will be a great challenge. The same approach can be used to generate other diseased animal models for pathological research and drug development in mouse, rabbit, dog, pig, sheep, cattle, monkey, and human.

Intron-Mediated Gene Silencing in Chicken Embryos

The in vivo model of chicken embryos has been widely utilized in many developmental biology, signal transduction and flu vaccine development research. We thus successfully tested the feasibility of transgene-like gene silencing in chicken in vivo using intronic RNA and discovered that a coupled interaction of nascent pre-mRNA transcription and intron excision occurring proximal to genomic perichromatin fibrils may be essential for microRNA (miRNA) biogenesis. The SpRNAi intron can be integrated into a host gene for transgenic expression. In an effort to examine such a transgenic model of intronic miRNA, we transfected chicken embryos with an isolated RCAS SpRNAi construct containing a hairpin anti-β-catenin pre-miRNA insert, which was directed against the protein-coding region of the chicken β-catenin gene sequence (NM205081). As an example, the β-catenin gene was selected because its products play a critical role in the biological development. β-catenin is known to be involved in the growth control of skin and liver tissues in chicken.

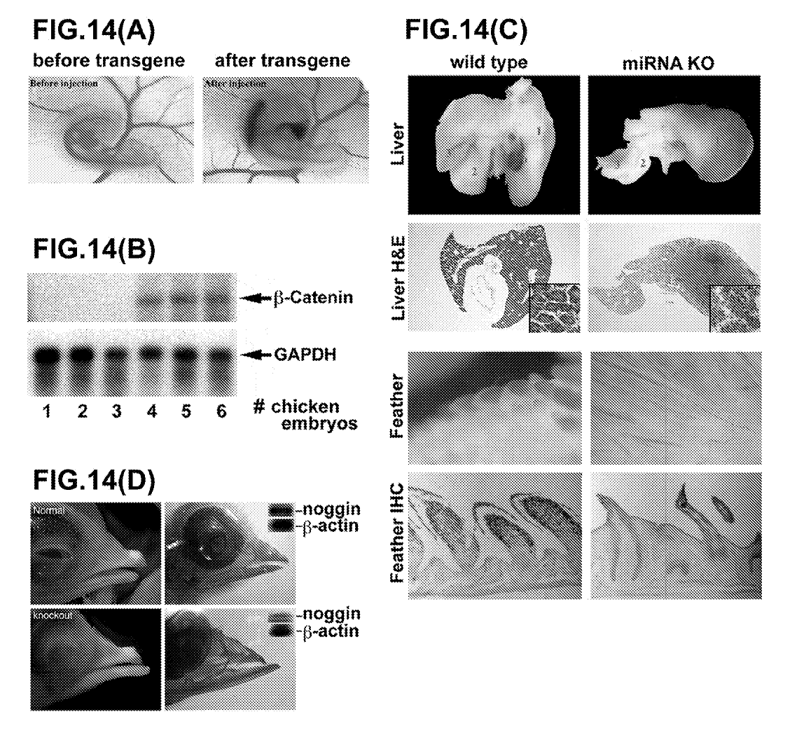

As shown in FIG. 14B, Northern blot analysis for the targeted β-catenin mRNA expression in the dissected livers showed that β-catenin expression in the wild-type control livers remained normal (lanes 4-6), whereas expression in the miRNA-treated samples was decreased dramatically (lanes 1-3). miRNA-mediated gene silencing degraded more than 98% of β-catenin mRNA expression in the embryonic chicken, but had no effect on the house-keeping gene GAPDH expression, indicating its high target specificity and very limited interferon-related cytotoxicity in vivo.

After ten days of primordial injection, the embryonic chicken livers showed an enlarged and engorged first lobe, but the size of the second and third lobes of the livers were dramatically decreased (FIG. 14C). Histological sections of normal livers showed hepatic cords and sinusoidal space with few blood cells. In the anti-β-catenin miRNA-treated embryos, the general architecture of the hepatic cells in lobes 2 and 3 remained unchanged; however, there were islands of abnormal regions in lobe 1. The endothelium development appeared to be defective and blood leaked outside of the blood vessels. Abnormal types of hematopoietic cells and cell precursors were also observed between the space of hepatocytes, particularly dominated by a population of small cells with round nuclei and scanty cytoplasm. In severely affected regions, hepatocytes were disrupted (FIG. 14C, small windows) and the diffused miRNA effect further inhibited the feather growth in the skin area close to the injection site. The results discussed above showed that the anti-β-catenin miRNA was very effective in knocking out the targeted gene expression at a very low dose and was effect over a long period of time (≧10 days). Further, the miRNA-mediated gene silencing effect appeared to be very specific to the target gene function, as off-targeted organs appear to be normal, indicating that intronic miRNA herein possessed no overt toxicity.

In another attempt to silence noggin expression in the chicken mandible beak area using a similar approach (FIG. 14D), an enlarged lower beak was observed, reminiscent of BMP4-overexpressing chicken embryos. Skeleton staining showed outgrowth of bone and cartilage tissues in the injected mandible area (FIG. 14D, right panels) and Northern blot analysis further confirmed that about 65% of noggin mRNA expression was knocked out in this region (small windows). Because bone morphogenetic protein 4 (BMP4) is known to promote bone development and since noggin is an antagonist of BMP2/4/7, this explains that SpRNAi-mediated noggin knockout chicken exhibited a morphological change similar to the BMP4-overexpression chicken described previously. Thus, gene silencing in chicken by SpRNAi transfection has a great potential of localized transgene-like manipulation in developmental biology.

Intron-Mediated Gene Silencing in Mouse Skins

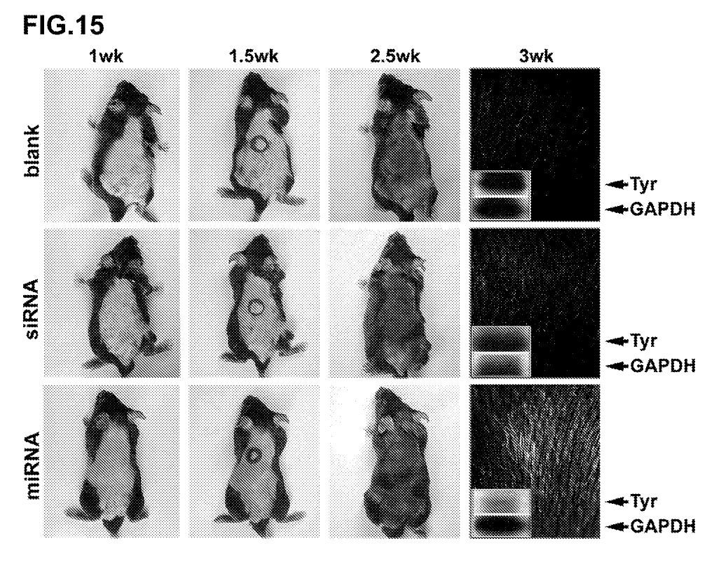

To test the intronic miRNA effect on adult mammals (FIG. 15), we used a vector-based miRNA delivery approach similar to the previously reported transgene-like method in chicken embryos. Patched albino (white) skins of melanin-knockdown mice (Rosa-26 black strain) were created by a succession of intra-cutaneous (i.c.) transduction of an anti-tyrosinase (Tyr) miRNA transgene construct (50 μg) for 4 days (total 200 μg). Tyr, a type-I membrane protein and copper-containing enzyme, catalyzes the critical and rate-limiting step of tyrosine hydroxylation in the biosynthesis of melanin (black pigment) in skins and hairs. After 14-day incubation, the production of melanin was blocked due to a loss of its intermediates resulted from the silencing effect of anti-Tyr miRNA. Contrarily, the blank control and the U6-directed siRNA/dsRNA-transfected mice presented normal skin color (black), indicating that miRNA rather than siRNA could trigger effective gene silencing against Tyr expression in mouse skins. Moreover, Northern blot analysis of mRNAs from hair follicles showed a 76.1±5.3% reduction of Tyr expression two days after transfection, in consistent with the immunohistochemical (IHC) staining results from the same skin area, whereas mild, non-specific degradation of common gene transcripts was detected in the siRNA-transfected skins (seen from smearing patterns of both house-keeping GAPDH and target Tyr mRNAs).

Thus, utilization of intronic miRNA expression vectors provides a powerful new strategy for in-vivo gene therapy, particularly for melanoma treatment. Under the same dosage, Pol-II-mediated miRNA did not cause detectable cytotoxicity, whereas Pol-III-directed siRNA induced non-specific mRNA degradation as previous reports (Sledz et. al. supra; Lin (2004b) supra). This underscores the fact that miRNA is effective in vivo without the cytotoxic effect of double-stranded RNA. This result also indicates that the miRNA gene silencing effect is stable and efficient in knocking down the target gene expression over a relatively long time since hair regrowth requires at least a ten-day period of time to reach full recovery. Advantageously, intronic miRNA offers relatively long-term, effective and safe gene manipulation in local animal tissues and organs, preventing the lethal effect of systemic gene knockouts used in the conventional transgenic animal models. The same approach and strategy can be used to increase milk production in cow, to increase meat production in cattle and pig, to generate big size animals or small size pets, and to develop large size as well as weather-resistant plants.

Anti-Viral Therapy Using Intron-Mediated Gene Silencing Against Foreign Transgenes

During the early HIV infection, the viral reverse transcriptase transcribes the HIV RNA genome into a double-stranded cDNA sequence, which forms a pre-integration complex with the matrix, integrase and viral protein R (Vpr). This complex is then transferred to the cell nucleus and integrated into the host chromosome, consequently establishing the HIV provirus. We hypothesized that, although HIV carries few reverse transcriptase and matrix proteins during its first entry into host cells, the co-suppression of Pr55^{gag} and p66/p51^{pol} gene expression by miRNA would eliminate the production of infectious viral particles in the late infection phase. Silencing Pr55^{gag} may prevent the assembly of intact viral particles due to the lack of matrix and capsid proteins, while suppression of protease in p66/p51^{pol} can inhibit the maturation of several viral proteins. HIV expresses about nine viral gene transcripts which encode at least 15 various proteins; thus, the separation of a polyprotein into individual functional proteins requires the viral protease activity.

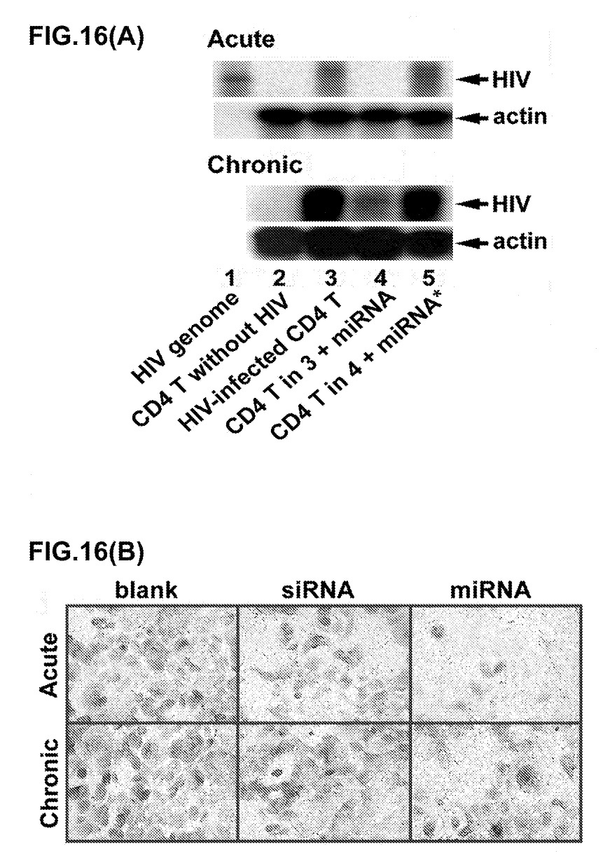

The anti-HIV SpRNAi-rGFP vector was tested in CD4+ T lymphocyte cells from HAART-treated, HIV-seropositive patients. Because only partial complementarity between miRNA and its target RNA is needed to trigger the gene silencing effect, this approach may be superior to current small molecule drugs since the high rate of HIV mutations often produces resistance to such agents. Northern blot analysis in FIG. 16A demonstrated the gene silencing effect of anti-HIV intronic miRNA transfection (n=3 for each set) on HIV-1 replication in CD4⁺ T lymphocytes from both acute and chronic phase AIDS patients. In the acute phase (≦one month), transfection of 50 nM anti-HIV SpRNAi-rGFP vector degraded an average of 99.8% viral RNA genome (lane 4), whereas the same treatment knocked down only an average of 71.4±12.8% viral genome replication in the chronic phase (about 2-year infection). Immunocytochemical staining for HIV p24 marker protein confirmed the results of Northern blot analysis (FIG. 16B).

Sequencing analysis has revealed at least two HIV-1 mutants in the acute phase and seven HIV-1 mutants in the chronic phase within the targeted HIV genome domain. It is likely that the higher genome complexity produced by HIV mutations in chronic infections reduces miRNA-mediated silencing efficacy. Transfection of 50 nM miRNA*-expressing vector containing homology to the HIV-1 genome however reverses the RNAi effect on viral genome, indicating the specificity of the SpRNAi-derived miRNA and miRNA* effects (lanes 4 and 5). Expression of cellular house-keeping gene, β-actin, was normal and showed no interferon-induced non-specific RNA degradation. These results suggest that the anti-HIV SpRNAi-rGFP vector is highly specific and efficient in suppressing HIV-1 replication in early infections. In conjunction with an intermittent interleukin-2 (IL-2) therapy, the growth of non-infected CD4⁺ T lymphocytes can be stimulated to eliminate the HIV-infected cells, demonstrating a very promising pharmaceutical and therapeutic approach for AIDS therapy. The same approach and strategy can be used for the development of vaccine and therapy against other viral infection, such as hepatitis B virus (HBV), hepatitis C virus (HCV), herpes virus (HPV), smallpox virus, flu virus and so on.

Recovery of miRNA-Suppressed Gene Expression by Antisense microRNA (miRNA*)

Our studies of SpRNAi utilities also demonstrated that an intron-mediated enhancement (IME) phenomenon, similar to that reported in plants, takes place in mammalian cells. Previous studies in Arabidopsis and Nicotiana spp., indicate that introns play an important role in posttranscriptional gene modulation for both enhancing and silencing specific gene expression. When certain intronic sequences were inserted into an intronless gene, the expression of such gene transcripts was steadily increased ranging from 2 fold to over 10 fold. The intron-mediated increase of gene expression is usually at the level of mRNA accumulation although its mechanism remains to be elucidated.

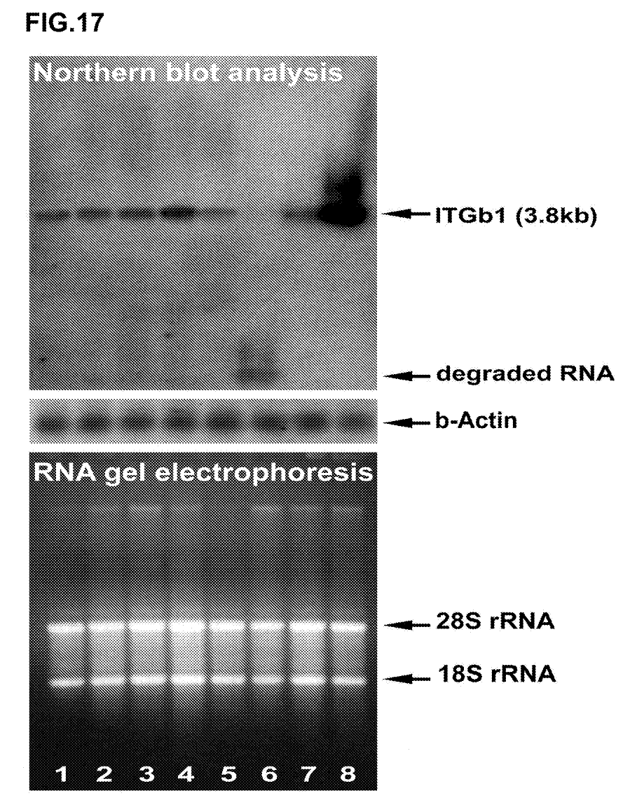

We have tested the transfection of SpRNAi-rGFP genes containing inserts homologous to the first in-frame intron at the nts 43˜68 region of integrin β1 (ITGb1) in human cervical cancer HeLa cells, which express a moderate amount of ITGb1 adhesion protein consistent with elevated cell proliferation and metastatic activity. The increase of ITGb1 expression potentially restricts the spreading of cervical cancer cells in situ because alterations of cellular characteristics were observed through the attachment of cancer cells to a glycine-coated culture dish surface. FIG. 17 shows strong over-expression of ITGb1 mRNA by transfection of SpRNAi-rGFP containing hairpin inserts homologous to ITGb1 intron 1 (lane 8, hairpin-ITGb1), whereas transfection of SpRNAi-rGFP containing either sense-strand (lane 4, sense-ITGb1) or antisense-strand (lane 5, antisense-ITGb1) inserts or co-transfection of both inserts at the equal concentration (lane 6, dsRNA-ITGb1) showed no gene enhancement effects as determined by Northern blot analysis. The co-transfection of dsRNA-ITGb1 actually resulted in a marked gene silencing effect potentially through short interfering double-stranded RNA (siRNA/dsRNA)-induced RNA interference (RNAi). Again, co-transfection of hairpin-ITGb1 and dsRNA-ITGb1 (lane 7) can neutralize mutual gene modulation effects, suggesting the incompatibility between IME and RNAi mechanisms competing for the same target gene. Other Northern blot results were shown from left to right: (lane 1) normal HeLa cells without transfection (blank control), (lane 2) HeLa cells transfected with empty SpRNAi-rGFP vector without any intronic insert (negative control), and (lane 3) HeLa cells transfected with SpRNAi-rGFP vector with anti-EGFP insert (off-target control).

A. Definitions

To facilitate understanding of the invention, a number of terms are defined below:

Nucleotide: a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. A nucleoside containing at least one phosphate group bonded to the 3′ or 5′ position of the pentose is a nucleotide.

Oligonucleotide: a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

Nucleic Acid: a polymer of nucleotides, either single or double stranded.

Nucleotide Analog: a purine or pyrimidine nucleotide that differs structurally from A, T, G, C, or U, but is sufficiently similar to substitute for the normal nucleotide in a nucleic acid molecule.

Gene: a nucleic acid whose nucleotide sequence codes for an RNA and/or a polypeptide (protein). A gene can be either RNA or DNA.

Base Pair (bp): a partnership of adenine (A) with thymine (T), or of cytosine (C) with guanine (G) in a double stranded DNA molecule. In RNA, uracil (U) is substituted for thymine. Generally the partnership is achieved through hydrogen bonding.

Precursor messenger RNA (pre-mRNA): primary ribonucleotide transcripts of a gene, which are produced by type-II RNA polymerase (Pol-II) machineries in eukaryotes through an intracellular mechanism termed transcription. A pre-mRNA sequence contains a 5′-end untranslated region, a 3′-end untranslated region, exons and introns.

Intron: a part or parts of a gene transcript sequence encoding non-protein-reading frames.

Exon: a part or parts of a gene transcript sequence encoding protein-reading frames.

Messenger RNA (mRNA): assembly of pre-mRNA exons, which is formed after intron removal by intranuclear spliceosomal machineries and served as a protein-coding RNA for protein synthesis.

cDNA: a single stranded DNA that is complementary to an mRNA sequence and does not contain any intronic sequences.

Sense: a nucleic acid molecule in the same sequence order and composition as the homologous mRNA. The sense conformation is indicated with a “+”, “s” or “sense” symbol.

Antisense: a nucleic acid molecule complementary to the respective mRNA molecule. The antisense conformation is indicated as a “−” symbol or with an “a” or “antisense” in front of the DNA or RNA, e.g., “aDNA” or “aRNA”.

5′-end: a terminus lacking a nucleotide at the 5′ position of successive nucleotides in which the 5′-hydroxyl group of one nucleotide is joined to the 3′-hydroyl group of the next nucleotide by a phosphodiester linkage. Other groups, such as one or more phosphates, may be present on the terminus.

3′-end: a terminus lacking a nucleotide at the 3′ position of successive nucleotides in which the 5′-hydroxyl group of one nucleotide is joined to the 3′-hydroyl group of the next nucleotide by a phosphodiester linkage. Other groups, most often a hydroxyl group, may be present on the terminus.

Template: a nucleic acid molecule being copied by a nucleic acid polymerase. A template can be single-stranded, double-stranded or partially double-stranded, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

Nucleic Acid Template: a double-stranded DNA molecule, double stranded RNA molecule, hybrid molecules such as DNA-RNA or RNA-DNA hybrid, or single-stranded DNA or RNA molecule.

Conserved: a nucleotide sequence is conserved with respect to a pre-selected (reference) sequence if it non-randomly hybridizes to an exact complement of the pre-selected sequence.

Complementry or Complementarity or Complementation: used in reference to polynucleotides (i.e., a sequence of nucleotides) related by the base-pairing rules. For example, the sequence “A-G-T” is complementary to the sequence “T-C-A,” and also to “T-C-U.” Complementation can be between two DNA strands, a DNA and an RNA strand, or between two RNA strands. Complementarity may be “partial” or “complete” or “total”. Partial complementarity or complementation occurs when only some of the nucleic acid bases are matched according to the base pairing rules. Complete or total complementarity or complementation occurs when the bases are completely matched between the nucleic acid strands. The degree of complementarity between nucleic acid strands has significant effects on the efficiency and strength of hybridization between nucleic acid strands. This is of particular importance in amplification reactions, as well as in detection methods that depend on binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

Homologous or Homology: refers to a polynucleotide sequence having similarities with a gene or mRNA sequence. A nucleic acid sequence may be partially or completely homologous to a particular gene or mRNA sequence, for example. Homology may also be expressed as a percentage determined by the number of similar nucleotides over the total number of nucleotides.

Complementry Bases: nucleotides that normally pair up when DNA or RNA adopts a double stranded configuration.

Complementry Nucleotide Sequence: a sequence of nucleotides in a single-stranded molecule of DNA or RNA that is sufficiently complementary to that on another single strand to specifically hybridize between the two strands with consequent hydrogen bonding.

Hybridize and Hybridization: the formation of duplexes between nucleotide sequences which are sufficiently complementary to form complexes via base pairing. Where a primer (or splice template) “hybridizes” with target (template), such complexes (or hybrids) are sufficiently stable to serve the priming function required by a DNA polymerase to initiate DNA synthesis. There is a specific, i.e. non-random, interaction between two complementary polynucleotides that can be competitively inhibited.

RNA interference (RNAi): a posttranscriptional gene silencing mechanism in eukaryotes, which can be triggered by small RNA molecules such as microRNA and small interfering RNA. These small RNA molecules usually function as gene silencers, interfering with expression of intracellular genes containing either completely or partially complementarity to the small RNAs.

MicroRNA (miRNA): single-stranded RNAs capable of binding to targeted gene transcripts that have partial complementarity to the miRNA. miRNA is usually about 16-28 oligonucleotides in length and is able to either directly degrade its intracellular mRNA target(s) or suppress the protein translation of its targeted mRNA, depending on the complementarity between the miRNA and its target mRNA. Natural miRNA molecules are found in almost all eukaryotes, functioning as a defense against viral infections and allowing regulation of gene expression during development of plants and animals.

MicroRNA* (miRNA*): single-stranded RNA containing partial or complete complementarity to the sequence of a mature microRNA.

Small interfering RNA (siRNA): short double-stranded RNAs sized about 18-25 perfectly base-paired ribonucleotide duplexes and capable of degrading target gene transcripts with almost perfect complementarity.

Short hairpin RNA (shRNA): single-stranded RNA that contains a pair of partially or completely matched stem-arm nucleotide sequences divided by an unmatched oligonucleotide loop to form a hairpin-like structure. Many natural miRNA products are derived from hairpin-like RNA precursors, namely precursor microRNA (pre-miRNA).

Vector: a recombinant nucleic acid molecule such as recombinant DNA (rDNA) capable of movement and residence in different genetic environments. Generally, another nucleic acid is operatively linked therein. The vector can be capable of autonomous replication in a cell in which case the vector and the attached segment is replicated. One type of preferred vector is an episome, i.e., a nucleic acid molecule capable of extrachromosomal replication. Preferred vectors are those capable of autonomous replication and/or expression of nucleic acids to which they are linked. Vectors capable of directing the expression of genes encoding for one or more polypeptides are referred to herein as “expression vectors”. Particularly important vectors allow cloning of cDNA from mRNA produced using a reverse transcriptase.

Cistron: a sequence of nucleotides in a DNA molecule coding for an amino acid residue sequence and including upstream and downstream DNA expression control elements.

Promoter: a nucleic acid to which a polymerase molecule recognizes, perhaps binds to, and initiates synthesis. For the purposes of the instant invention, a promoter can be a known polymerase binding site, an enhancer and the like, any sequence that can initiate synthesis by a desired polymerase.

Antibody: a peptide or protein molecule having a pre-selected conserved domain structure coding for a receptor capable of binding a pre-selected ligand.

B. Compositions

A recombinant nucleic acid composition for inducing intron-mediated gene silencing comprises:

(1) At least an intron, wherein said intron is flanked with a plurality of exons and can be cleaved out of the exons by intracellular RNA splicing and/or processing machineries; and

(2) A plurality of exons, wherein said exons can be linked to form a gene possessing a desired function.

The above recombinant nucleic acid composition, further comprises:

(1) At least a restriction/cloning site, wherein said restriction/cloning site is used for inserting the recombinant nucleic acid composition into an expression-competent vector for expressing the RNA transcript of said recombinant nucleic acid composition in a desired cell or organism; and

(2) A plurality of transcription and translation termination sites, wherein said transcription and translation termination sites are used for produce the correct RNA transcript sizes of said recombinant nucleic acid composition.

The intron of the above recombinant nucleic acid composition, further comprises:

(1) A gene-specific insert complementary or homologous to a targeted gene;

(2) A splicing donor site;

(3) A splicing acceptor site;

(4) A branch point domain for spliceosomal recognition;

(5) At least a poly-pyrimidine tract for spliceosomal interaction; and

(6) linkers for connection of the above major components.

The splicing donor site is a nucleotide motif homologous to (5′-exon-AG)-(splicing point)-GTA(A/-)GAG(G/T)-3′ sequences (SEQ.ID.NO.1), including but not limited, 5′-AG GTAAGAGGAT-3′, 5′-AG GTAAGAGT-3′, 5′-AG GTAGAGT-3′, and 5′-AG GTAAGT-3′ etc. The splicing acceptor site is another kind of a nucleotide motif homologous to 5′-G(W/-)(T/G)(C/G)C(T/C)(G/A)CAG-(splicing point)-(G/C-3′-exon) sequences (while W is a pyrimidine A, T or U) (SEQ.ID.NO.2), including but not limited, 5′-GATATCCTGCAG G-3′, 5′-GGCTGCAG G-3′, and 5′-CCACAG C-3′ etc. The branch point is an “A” nucleotide located within a sequence homologous to 5′-TACT(A/T)A*(C/T)(-/C)-3′ (while the symbol “*” marks the branch site) (SEQ.ID.NO.3), including but not limited, 5′-TACTAAC-3′, 5′-TACTTATC-3′ and so on. The poly-pyrimidine tract is a high T or C content oligonucleotide sequence homologous to 5′-(TY)m(C/-)(T)nC(C/-)-3′ or 5′-(TC)nNCTAG(G/-)-3′ (while Y is a C or T/U and the “-” means an empty site). The symbols of “m” and “n” indicates multiple repeats ≧1; most preferably, m=1˜3 and n=7˜12. For all the above spliceosomal recognition components, the deoxythymidine (T) is replaceable with a uridine (U).

C. Methods

A method for inducing intron-mediated gene silencing effects comprises:

(1) Constructing a recombinant nucleic acid composition containing at least an intron flanked with a plurality of exons, wherein said intron can be cleaved out of the exons by RNA splicing and/or processing for gene silencing and said exons can be linked together to form a mature gene transcript with or without a desired function;

(2) Cloning said recombinant nucleic acid composition into an expression-competent vector;

(3) Introducing said vector into a cell or an organism;

(4) Generating RNA transcript of said recombinant nucleic acid composition; and

(5) Releasing the functional part(s) of said intron via intracellular RNA splicing and/or processing mechanisms, so as to provide gene silencing effects against a targeted gene or genes containing sequences complementary to said intron.

Alternatively, a method for inducing RNA interference and/or posttranscriptional gene silencing effects comprises:

(1) Constructing a recombinant gene containing a functional RNA polymerase promoter and at least an intron flanked with a plurality of exons, wherein said intron can be cleaved out of the exons by RNA splicing and/or processing for gene silencing and said exons can be linked together to form a mature gene transcript with or without a desired function;

(2) Introducing said recombinant gene into a cell or an organism;

(3) Generating RNA transcript of said recombinant gene; and

(4) Releasing the functional parts of said intron via RNA splicing and/or processing mechanisms, so as to provide gene silencing effects against a targeted gene or genes containing sequences complementary to said intron.

More broadly, a method for suppressing gene function or silencing gene expression, comprising the steps of:

(1) providing: i) a substrate expressing a targeted gene, and ii) a nucleic acid composition comprising a recombinant gene capable of producing a specific RNA transcript, which is in turn able to generate pre-designed gene silencing molecules through intracellular RNA splicing and/or processing mechanisms to inhibit the targeted gene expression or suppress the targeted gene function in the substrate;

(2) treating the substrate with the nucleic acid composition under conditions such that the targeted gene expression or function in the substrate is inhibited.

Various changes and modifications obvious to one skilled in the art to which the present invention pertains are deemed to be within the spirit, scope and contemplation of the present invention as further defined in the appended claims.

### EXAMPLES

The following examples serve to illustrate certain preferred embodiments and aspects of the present invention and are not to be construed as limiting the scope thereof.

In the experimental disclosure which follows, the following abbreviations apply: M (molar); mM (millimolar); μm (micromolar); mol (moles); pmol (picomolar); gm (grams); mg (milligrams); μg (micrograms); ng (nanograms); L (liters); ml (milliliters); μl (microliters); ° C. (degrees Centigrade); cDNA (copy or complementary DNA); DNA (deoxyribonucleic acid); ssDNA (single stranded DNA); dsDNA (double-stranded DNA); dNTP (deoxyribonucleotide triphosphate); RNA (ribonucleic acid); PBS (phosphate buffered saline); NaCl (sodium chloride); HEPES (N-2-hydroxyethylpiperazine-N-2-ethanesulfonic acid); HBS (HEPES buffered saline); SDS (sodium dodecylsulfate); Tris-HCl (tris-hydroxymethylaminomethane-hydrochloride); and ATCC (American Type Culture Collection, Rockville, Md.).

### Example 1

### Cell Culture and Treatments

Rat neuronal stem cell clones AP31 and PZ5a were primary cultured and maintained as described by Palmer et. al., (J. Neuroscience, 1999). The cells were grown on polyornathine/laminin-coated dishes in DMEM/F-12 (1:1; high glucose) medium containing 1 mM L-glutamine supplemented with 1×N2 supplements (Gibco/BRL, Gaithersburg, Md.) and 20 ng/ml FGF-2 (Invitrogen, Carlsbad, Calif.), without serum at 37° C. under 5% CO₂. For long-term primary cultures, 75% of the medium was replaced with new growth medium every 48 hr. Cultures were passaged at ˜80% confluency by exposing cells to trypsin-EDTA solution (Irvine Scientific) for 1 min and rinsing once with DMEM/F-12. Detached cells were replated at 1:10 dilution in fresh growth medium supplemented with 30% (v/v) conditioned medium which had exposed to cells for 24 hr before passaging. Human prostatic cancer LNCaP cells were obtained from the American Type Culture Collection (ATCC) and grown in RPMI 1640 medium supplemented with 10% fetal bovine serum with 100 μg/ml gentamycin at 37° C. under 10% CO₂. The LNCaP culture was passaged at 80% confluency by exposing cells to trypsin-EDTA solution for 1 min and rinsing once with RPMI, and detached cells were replated at 1:10 dilution in fresh growth medium. After 48-hour incubation, RNA from tested cells was isolated by RNeasy spin columns (Qiagen, Valencia, Calif.), fractionated on a 1% formaldehyde-agarose gel, and transferred onto nylon membranes. The genomic DNA was also isolated by apoptotic DNA ladder kit (Roche Biochemicals, Indianapolis, Ind.) and assessed by 2% agarose gel electrophoresis, while cell growth and morphology were examined by microscopy and cell counting.

### Example 2

### SpRNAi-Containing Recombinant Gene Construction

Synthetic nucleic acid sequences used for generation of three different SpRNAi introns containing either sense-, antisense- or hairpin-eGFP insert were listed as followings: N1-sense, 5′-pGTAAGAGGAT CCGATCGCAG GAGCGCACCA TCTTCTTCAA GA-3′ (SEQ.ID.NO.4); N1-antisense, 5′-pCGCGTCTTGA AGAAGATGGT GCGCTCCTGC GATCGGATCC TCTTAC-3′ (SEQ.ID.NO.5); N2-sense, 5′-pGTAAGAGGAT CCGATCGCTT GAAGAAGATG GTGCGCTCCT GA-3′ (SEQ.ID.NO.6); N2-antisense, 5′-pCGCGTCAGGA GCGCACCATC TTCTTCAAGC GATCGGATCC TCTTAC-3′ (SEQ.ID.NO.7); N3-sense, 5′-pGTAAGAGGAT CCGATCGCAG GAGCGCACCA TCTTCTTCAA GTTAACTTGA AGAAGATGGT GCGCTCCTGA-3′ (SEQ.ID.NO.8); N3-antisense, 5′-pCGCGTCAGGA GCGCACCATC TTCTTCAAGT TAACTTGAAG AAGATGGTGC GCTCCTGCGA TCGGATCCTC TTAC-3′ (SEQ.ID.NO.9); N4-sense, 5′-pCGCGTTACTA ACTGGTACCT CTTCTTTTTT TTTTTGATAT CCTGCAG-3′ (SEQ.ID.NO.10); N4-antisense, 5′-pGTCCTGCAGG ATATCAAAAA AAAAAGAAGA GGTACCAGTT AGTAA-3′ (SEQ.ID.NO.11). Additionally, two exon fragments were generated by DraII restriction enzyme cleavage of red fluorescent rGFP gene (SEQ.ID.NO.12) at its 208th nucleotide (nt) site and the 5′ fragment was further blunt-ended by T4 DNA polymerase. The rGFP referred to a new red-fluorescin chromoprotein generated by insertion of an additional aspartate at the 69th amino acid (aa) of HcRed1 chromoproteins from Heteractis crispa, developing less aggregate and almost twice intense far-red fluorescent emission at the ˜570-nm wavelength. This mutated rGFP gene sequence was cloned into pHcRed1-N1/1 plasmid vector (BD Biosciences, Palo Alto, Calif.) and propagated with E. coli DH5α LB-culture containing 50 μg/ml kanamycin (Sigma Chemical, St. Louis, Mo.). We cleaved the pHcRed1-N1/1 plasmid with XhoI and XbaI restriction enzymes and purified a 769-bp rGFP fragment and a 3,934-bp empty plasmid separately from 2% agarose gel electrophoresis.

Hybridization of N1-sense to N1-antisense, N2-sense to N2-antisense, N3-sense to N3-antisense and N4-sense to N4-antisense was separately performed by heating each complementary mixture of sense and antisense (1:1) sequences to 94° C. for 2 min and then 70° C. for 10 min in 1×PCR buffer (e.g. 50 mM Tris-HCl, pH 9.2 at 25° C., 16 mM (NH₄)₂SO₄, 1.75 mM MgCl₂). Continuously, sequential ligation of either N1, N2 or N3 hybrid to the N4 hybrid was performed by gradually cooling the mixture of N1-N4, N2-N4 or N3-N4 (1:1) hybrids respectively from 50° C. to 10° C. over a period of 1 hr, and then T4 ligase and relative buffer (Roche) were mixed with the mixture for 12 hr at 12° C., so as to obtain introns for insertion into exons with proper ends. After the rGFP exon fragments were added into the reaction (1:1:1), T4 ligase and buffer were adjusted accordingly to reiterate ligation for another 12 hr at 12° C. For cloning the right sized recombinant rGFP gene, 10 ng of the ligated nucleotide sequences were amplified by PCR with rGFP-specific primers 5′-dCTCGAGCATG GTGAGCGGCC TGCTGAA-3′ (SEQ.ID.NO.13) and 5′-dTCTAGAAGTT GGCCTTCTCG GGCAGGT-3′ (SEQ.ID.NO.14) at 94° C., 1 min, 52°, 1 min and then 68° C., 2 min for 30 cycles. The resulting PCR products were fractionated on a 2% agarose gel, and a ˜900-bp nucleotide sequences was extracted and purified by gel extraction kit (Qiagen). The composition of this ˜900 bp SpRNAi-containing rGFP gene was further confirmed by sequencing.

Because the recombinant gene possessed an XhoI and an XbaI restriction site at its 5′- and 3′-end, respectively, it can be easily cloned into a vector with corresponding ends complementary to the XhoI and XbaI cloning sites. The vector was an expressing-capable organism or suborganism selected from the group consisted of plasmids, cosmids, phagmids, yeast artificial chromosomes, jumping genes, transposons and viral vectors. Moreover, since the insert within the intron was flanked with a PvuI and an MluI restriction site at its 5′- and 3′-end, respectively, we can remove and replace the insert with another different insert sequence possessing corresponding ends complementary to the PvuI and MluI cloning sites. The insert sequence was homologous or complementary to a gene fragment selected from the group consisted of fluorescent protein (GFP) genes, luciferase genes, lac-Z genes, viral genes, bacterial genes, plant genes, animal genes and human genes. The homology and/or complementarity rate is ranged from about 30˜100%, more preferably 35˜49% for a hairpin-like shRNA insert and 90˜100% for both sense-stRNA and antisense-siRNA inserts.

### Example 3

### Vector Cloning of SpRNAi-Containing Genes

For cloning into plasmids, since the SpRNAi-recombinant rGFP gene possessed an XhoI and an XbaI restriction site at its 5′- and 3′-end, respectively, it can be easily cloned into a vector with relatively complementary ends to the XhoI and XbaI cloning sites. We mixed the SpRNAi-recombinant rGFP gene and the linearized 3,934-bp empty pHcRed1-N1/1 plasmid at 1:16 (w/w) ratio, cooled the mixture from 65° C. to 15° C. over a period of 50 min, and then added T4 ligase and relative buffer accordingly into the mixture for ligation at 12° C. for 12 hr. This formed an SpRNAi-recombinant rGFP-expressing plasmid (SpRNAi-rGFP) vector, which can be propagated in E. coli DH5a LB-culture containing 50 μg/ml kanamycin. A positive clone was confirmed by PCR reaction with rGFP-specific primers SEQ.ID.NO.13 and SEQ.ID.NO.14 at 94° C., 1 min and then 68° C., 2 min for 30 cycles, and further sequencing. For cloning into viral vectors, the same ligation procedure was performed except using an XhoI/XbaI-linearized pLNCX2 retroviral vector (BD) instead. Since the insert within the SpRNAi intron was flanked with a PvuI and a MluI restriction site at its 5′- and 3′-end, respectively, we removed and replaced the eGFP insert with various integrin β1-specific insert sequences possessing corresponding ends complementary to the PvuI and MluI cloning sites.

Synthetic nucleic acid sequences used for generation of various SpRNAi introns containing either sense-, antisense- or hairpin-integrin β1 insert were listed as followings: P1-sense, 5′-pCGCAAGCAGG GCCAAATTGT GGGTA-3′ (SEQ.ID.NO.15); P1-antisense, 5′-pTAGCACCCAC AATTTGGCCC TGCTTGTGCG C-3′ (SEQ.ID.NO.16); P2-sense, 5′-pCGACCCACAA TTTGGCCCTG CTTGA-3′ (SEQ.ID.NO.17); P2-antisense, 5′-pTAGCCAAGCA GGGCCAAATT GTGGGTTGCG C-3′ (SEQ.ID.NO.18); P3-sense, 5′-pCGCAAGCAGG GCCAAATTGT GGGTTTAAAC CCACAATTTG GCCCTGCTTG A-3′ (SEQ.ID.NO.19); P3-antisense, 5′-pTAGCACCCAC AATTTGGCCC TGCTTGAATT CAAGCAGGGC CAAATTGTGG GTTGCGC (SEQ.ID.NO.20). These inserts were designed using Gene Runner software v3.0 (Hastings, Calif.) and formed by hybridization of P1-sense to P1-antisense, P2-sense to P2-antisense and P3-sense to P3-antisense, targeting the 244˜265th-nt sequence of integrin P1 gene (NM 002211.2). The SpRNAi-containing rGFP-expressing retroviral vector can be propagated in E. coli DH5a LB-culture containing 100 μg/ml ampcillin (Sigma). We also used a packaging cell line PT67 (BD) for producing infectious, replication-incompetent virus. The transfected PT67 cells were grown in DMEM medium supplemented with 10% fetal bovine serum with 4 mM L-glutamine, 1 mM sodium pyruvate, 100 μg/ml streptomycin sulfate and 50 μg/ml neomycin (Sigma) at 37° C. under 5% CO₂. The titer of virus produced by PT67 cells was determined to be at least 106 cfu/ml before transfection.

### Example 4

### Northern Blot Analysis

RNA (20 μg of total RNA or 2 μg poly[A⁺] RNA) was fractionated on 1% formaldehyde-agarose gels and transferred onto nylon membranes (Schleicher & Schuell, Keene, N.H.). A synthetic 75-bp probe (5′-dCCTGGCCCCC TGCTGCGAGT ACGGCAGCAG GACGTAAGAG GATCCGATCG CAGGAGCGCA CCATCTTCTT CAAGT-3′ (SEQ.ID.NO.21)) targeting the junction region between rGFP and the anti-eGFP insert was labeled with the Prime-It II kit (Stratagene, La Jolla, Calif.) by random primer extension in the presence of [³²P]-dATP (>3000 Ci/mM, Amersham International, Arlington Heights, Ill.), and purified with 30 bp-cutoff Micro Bio-Spin chromatography columns (Bio-Rad, Hercules, Calif.). Hybridization was carried out in the mixture of 50% freshly deionized formamide (pH 7.0), 5× Denhardt's solution, 0.5% SDS, 4×SSPE and 250 mg/mL denatured salmon sperm DNA fragments (18 hr, 42° C.). Membranes were sequentially washed twice in 2×SSC, 0.1% SDS (15 min, 25° C.), and once in 0.2×SSC, 0.1% SDS (15 min, 25° C.) before autoradiography.

### Example 5

### Suppression of Specific Protein Expression Levels

For interference of eGFP gene expression, we transfected rat neuronal stem cells with SpRNAi-rGFP plasmid vectors encoding either sense, antisense or hairpin anti-eGFP insert, using a Fugene reagent (Roche). Plasmids containing insert-free rGFP gene and SpRNAi-recombinant rGFP gene with an insert against HIV gag-p24 were used as negative control. Cell morphology and fluorescence imaging was photographed at 0-, 24- and 48-hour time points after transfection. At the 48-hr incubation time point, the rGFP-positive cells were sorted by flow cytometry and collected for Western blot analysis. For interference of integrin β1 expression, we transfected LNCaP cells with pLNCX2 retroviral vectors containing various SpRNAi-rGFP genes directed against the 244˜265th-nt domain of integrin β1 using the Fugene reagent. The transfection rate of pLNCX2 retroviral vector into LNCaP cells was tested to be about 20%, while the pLNCX2 virus was less infectious to LNCaP cells. The same analyses were performed as aforementioned.

### Example 6

### SDS-PAGE and Western Blot Analysis

For immunoblotting, cells were rinsed with ice cold PBS after growth medium was removed, and then treated with the CelLytic-M lysis/extraction reagent (Sigma) supplemented with protease inhibitors, Leupeptin, TLCK, TAME and PMSF, following manufacture's recommendations. The cells were incubated at room temperature on a shaker for 15 min, scraped into microtubes, and centrifuged for 5 min at 12,000×g to pellet the cell debris. Protein-containing cell lysate were collected and stored at −70° C. until use. Protein determinations were measured with SOFTmax software package on an E-max microplate reader (Molecular Devices, Sunnyvale, Calif.). Each 30 μg cell lysate was added into SDS-PAGE sample buffer either with (reduced) or without (unreduced) 50 mM DTT, and boiled for 3 min before loaded onto 8% polyacylamide gels, while the reference lane was loaded with 2˜3 μl molecular weight markers (Bio-Rad). SDS-polyacrylamide gel electrophoresis was performed according to the standard protocols (Molecular Cloning, 3rd ED). Protein fractionations were electroblotted onto a nitrocellulose membrane, blocked with Odyssey blocking reagent (Li-Cor Biosciences, Lincoln, Nebr.) for 1˜2 hr at the room temperature. We assessed GFP expression using primary antibodies directed against eGFP (1:5,000; JL-8, BD) or rGFP (1:10,000; BD), overnight at 4° C. The blot was then rinsed 3 times with TBS-T and exposed to a secondary antibody, goat anti-mouse IgG conjugate with Alexa Fluor 680 reactive dye (1:2,000; Molecular Probes), for 1 hr at the room temperature. After three more TBS-T rinses, scanning and image analysis were completed with Li-Cor Odyssey Infrared Imager and Odyssey Software v.10 (Li-Cor). For integrin β1 analysis, the same procedure was performed except using primary antibodies directed against integrin β1 (1:2,000; LM534, Chemicon, Temecula, Calif.).

### Example 7

### Combinational Therapy for AIDS Vaccination

To clone the SpRNAi-rGFP recombinant genes into viral vectors, the same ligation procedure was performed using pLNCX2 retroviral vector (BD) as described in Example 3. Because the intronic insert in SpRNAi was flanked with a PvuI and an MluI restriction site at the 5′- and 3′-ends, respectively, we can remove and replace the anti-eGFP inserts with a different anti-gene insert possessing cohesive ends to the PvuI and MluI restriction site. The inserts were designed using Gene Runner software v3.0 (Hastings, Calif.) and formed by hybridization of each pair of sense and antisense synthetic oligonucleotides, targeting the first exon sequence of either Naf1β (AJ011896), Nb2HP(H12458) or Tax1BP (U25801) gene. The SpRNAi-rGFP-expressing retroviral vector, namely SpRNAi-pLNCX2, was propagated in E. coli DH5α LB-culture containing 100 μg/ml ampcillin (Sigma). We can also use a PT67 packaging cell line (BD) for producing infectious, replication-incompetent virus. The transfected PT67 cells were grown in DMEM medium supplemented with 10% fetal bovine serum (BSA) with 4 mM L-glutamine, 1 mM sodium pyruvate, 100 μg/ml streptomycin sulfate and 50 μg/ml neomycin (Sigma) at 37° C. under 5% CO₂. The titer of virus produced by PT67 cells was determined to be over 5×10⁶ cfu/ml before transfection.

CD4⁺ T lymphocytes were isolated from peripheral blood mononuclear cells of normal donors using immunomagnetic beads (Miltenyi Biotec, Auburn, Calif.) and cultured in RPMI 1640 medium supplemented with 20% BSA, 4 μg/ml phytohemagglutinin and 50 U/ml recombinant human IL-2 (Roche) at 37° C., 10% CO₂. For anti-HIV vaccination, SpRNAi-pLNCX2 provirus (˜6×10⁶ cfu/ml) in 100 μl of RPMI 1640 medium was applied to 2 ml medium (about 30 nM) in suspension flasks containing ˜800 T cells/μl. One day prior to infection, the culture medium was replaced with medium containing 2% fetal bovine serum and 200 U/ml IL-2 for 30 hr. After that, to establish HIV infection, the T cells with or without SpRNAi-pLNCX2 transfection (˜150 cell/μl) were mixed with supernatants collected from pooled HIV-seropositive T cell extracts from HAART-treated patients. Viral supernatants contained ˜3×10⁵ total viral RNA copies/ml, approximate to FDA standards established for appearance of AIDS symptoms. Infection occurred at an MOI of 0.1. Viral stock solutions confirmed to Virology Quality Assurance Standards for infection and were diluted in plasma collected from HIV-seronegative donors. Viral aliquotes were stored at −80° C. until needed for infection. ELISA detection (Roche; FIG. 10B) of HIV gag-p24 marker (Chemicon) was performed following manufactures' protocols and compared to Northern blotting results (FIG. 10A). Northern blotting was performed as described in Example 4, except using an isotope-labeled anti-gag/p24 probe.

### Example 8

### Generation of SpRNAi-Recombinant Gene Templates Using RNA-PCR

The RNA-polymerase cycling reaction (RNA-PCR) procedure can be modified to synthesize mRNA-cDNA, DNA-aRNA, DNA-cDNA and mRNA-aRNA duplex hybrids as transgenes, isolated from an SpRNAi recombinant gene, the template of an expression-competent vector or a transcriptome source (Lin et. al. (1999) Nucleic Acids Res. 27: 4585-4589). As an example of using the SpRNAi recombinant gene as a source, a SpRNAi-pLNCX2 recombinant gene vector containing homologues to HIV-1 genome from +2113 to +2453 bases was generated following a procedure similar to Example 2. The RNA products (10˜50 μg) of the anti-HIV SpRNAi-pLNCX2 recombinant gene were transcribed from about 10⁶ transfected cells, isolated by RNeasy columns (Qiagen) and then continuously hybrid to its synthetic complementary DNA (cDNA) by heating and then cooling the mixture from 65° C. to 15° C. over a period of 50 min. Transfection was completed following the same procedure shown in Example 5.

### Example 9

### Intron-Mediated Gene Silencing in Zebrafish

Tg(UAS:gfp) strain zebrafish were raised in a fish container with 10 ml of 0.2× serum-free RPMI 1640 medium during transfection. A transfection pre-mix was prepared by gently dissolving 6 μl of a Fugene liposomal transfection reagent (Roche Biochemicals, Indianapolis, Ind.) in 1× serum-free RPMI 1640 medium. SpRNAi-rGFP vectors (20 μg) as described in Example 3 were then mixed with the pre-mix for 30 min and directly applied to the Tg(UAS:gfp) fish. Total three dosages were given in a 12 hr interval (total 60 μg). Samples were collected 60 hr after the first transfection and analyzed by a microscopic quantitation system (Nikon 80i fluorescent imaging; FIG. 12B) as well as Western blot analysis (FIG. 12C). Western blotting was performed as described in Example 6.

### Example 10

### Generation of Transgenic Zebrafish Using SpRNAi-rGFP Vectors

Zebrafish possesses three FMRP-related genes fmr1, fxr1 and fxr2, which are orthologous to the human FMR1, FXR1 and FXR2 genes, respectively. The expression patterns of these FMRP-familial genes in zebrafish tissues are broadly consistent with those in mouse and human, suggesting that such a loss-of-fmr1-function zebrafish is an excellent model organism for studying the etiology of fragile X mental retardation. We constructed the anti-fmr1 miRNA transgene based on a proof-of-principle design of the SpRNAi-rGFP vector as previously described in the generation of gene-knockout zebrafish (Examples 3 and 9). Because the intronic insert in SpRNAi was flanked with a PvuI and a MluI restriction site at its 5′- and 3′-end respectively, we can remove and replace the eGFP insert with various anti-fmr1 insert sequences possessing relatively cohesive ends to the PvuI and MluI sites. The intronic pre-miRNA insert in this SpRNAi-rGFP vector construct is directed against the nt 25-45 region of the zebrafish fmr1 5′-UTR methylation site (accession number NM152963). This target region contains several 5′-UTR (CGG) repeats, reminiscent of the native anti-FMR1 miRNA target site in human fragile X syndrome.

The anti-fmr1 SpRNAi-rGFP vector was further transfected into 12-hour postfertilization (hpf)-stage zebrafish embryos using the Fugene reagent following the same protocol as described in Example 9 (Lin et al. (2005) supra). The miRNA was expressed under the control of a GABA(A) receptor βZ2 gene promoter in zebrafish brain. After 72-hr post-transfection, zebrafish larvae with the same treatment were homogenized and lyzed using the CelLytic-M lysis/extraction reagent (Sigma Chemicals). Cell lysates were then used in Western blot analysis (FIG. 13A) to determine the levels of fish fmr1 protein with a monoclonal anti-FMR1 IgG antibody (Chemicon), following manufacture's suggestions. The gene-knockdown efficacy is determined by total amounts (in ratio) of the fmr1 protein in whole zebrafish larvae (line chart). Pallium neuron morphology was changed after fmr1-knockout, reminiscent of dendritic neurons in human fragile X syndrome (FIG. 13B).

### Example 11

### Intron-Mediated Gene Silencing in Chicken Embryos

Because the intronic insert in SpRNAi was flanked with a PvuI and a MluI restriction site at its 5′- and 3′-end, respectively, we can change the intronic insert with various anti-gene sequences possessing cohesive ends to the PvuI and MluI sites. We thus transfected chicken embryos with an isolated SpRNAi construct containing a hairpin anti-β-catenin pre-miRNA insert, which was directed against the protein-coding region of the chicken β-catenin gene sequence (NM205081). Using embryonic day 3 chicken embryos, a dose of 25 nM of the isolated SpRNAi construct was injected into the body region close to where the liver primordia would form (FIG. 14A). For efficient delivery into target tissues, the construct was mixed with the Fugene reagent (Roche) as described in Example 9. A 10% (v/v) fast green solution was concurrently added during the injection as a dye indicator. The mixtures were injected into the ventral side near the liver primordia below the heart using heat pulled capillary needles. After injection, the embryonic eggs were sealed with sterilized scotch tapes and incubated in a humidified incubator at 39-40° C. till day 12 when the embryos were examined and photographed under a dissection microscope. The specific β-catenin gene silencing results were confirmed by Northern blot analysis (FIG. 14B). Several deformities were observed in the targeted organs, e.g. liver (FIG. 14B), while the embryos still survived and there was no visible overt toxicity or overall perturbation of embryo development.

### Example 12

### Intron-Mediated Gene Silencing in Mouse In Vivo

Patched albino (white) skins of melanin-knockdown mice (Rosa-26 black strain) were created by a succession of intra-cutaneous (i.c.) transduction of an isolated SpRNAi-transgene construct directed against the tyrosinase (Tyr) gene for 4 days (total 200 μg). This SpRNAi-transgene construct was designed as described in Examples 3 and 8, except using a hairpin anti-Tyr pre-miRNA insert instead. For efficient delivery into target tissues, the construct was mixed with the Fugene reagent (Roche) following the same protocol as described in Example 9. The gene silencing results were further confirmed by Northern blot analysis as shown in FIG. 15, small windows. Northern blotting was performed as described in Example 4, except using an antisense probe directed against Tyr nt 183-302.

### Example 13

### Anti-HIV Therapy Using Multiple Intronic RNAs

The following experimentation demonstrates suppression of exogenous retrovirus replication in patient-extracted CD4+ T lymphocytes using the present invention. Specific anti-HIV SpRNAi-rGFP vectors were designed to directly target against the gag-pol region from approximately nts +2113 to +2450 of the HIV-1 genome. This region is relatively conserved and can serve as a good target for anti-HIV treatment. The viral genes located in this target region include 3′-proximal Pr₅₅^{gag} polyprotein (i.e., matrix p17+capsid p24+nucleocapsid p7) and 5′-proximal p66/p51^{pol} polyprotein (i.e., protease p10+reverse transcriptase); all these components have critical roles in viral replication and infectivity.

In order to test the feasibility of using intronic RNA fragments directed against multiple HIV gene targets, the SpRNAi-pLNCX2 proviral vector shown in Example 7 was re-designed to target an early-stage gene locus containing gag/pol/pro viral genes and p24 HIV gene marker. Expectedly, the anti-gag/pol/pro SpRNAi-pLNCX2 transfection will interfere the integration of viral provirus into host chromosome and also to prevent the activation of several viral genes, while the anti-p24 effect will provide a visual indicator for detecting viral activity determined by an ELISA or immunocytochemical (IHC) staining assay. The results showed that such strategy was effective in knocking out exogenous viral gene expression ex vivo in a CD4⁺ T lymphocyte extract model. Peripheral blood mononuclear cells (PBMC) extracted from patients were purified by CD4⁺-affinity immunomagnetic beads and grown in RPMI 1640 medium with 200 U/ml IL-2 adjuvant treatment for more than two weeks. An intronic SpRNAi insert containing partial HIV genomic sequence from +2113 to +2453 bases was generated from the SpRNAi-recombinant gene as described in Example 8. After 96 hr incubation, the expression activity of HIV-1 genome was measured by Northern blot analysis (FIG. 16A). IHC staining for HIV p24 marker protein was used here to confirm the results of Northern blot analysis (FIG. 16B).

### Example 14

### Recovery of miRNA-Targeted Gene Expression by miRNA*

We transfected human HeLa cells with SpRNAi-rGFP plasmid vectors containing various pre-miRNA* inserts directed against integrin β1 intron 1 nt 43˜68, using the Fugene reagent (Roche), as described in Example 9. HeLa cells, a cervical cancer cell line acquired from ATCC, were grown in DMEM medium supplemented with 10% fetal calf serum at 37° C. with 5% CO₂. Thirty-six hours after transfection, total RNAs were extracted using RNeasy spin columns (Qiagen), fractionated on 1% formaldehyde-agarose gels and transferred onto nylon membranes (Schleicher & Schuell). Northern blot analysis was performed as aforementioned in Example 4, except using probes specific for integrin β1 (ITGb1).

### REFERENCES

The following references are hereby incorporated by reference as if fully set forth herein:
(1) Elbashir et. al. (2001) Duplexes of 21-nucleotide RNAs mediate NA interference in cultured mammalian cells. Nature 411: 494-498.
(2) Lin et. al. (2001) A novel mRNA-cDNA interference phenomenon for silencing bcl-2 expression in human LNCaP cells. Biochem. Biophys. Res. Commun. 281: 639-644.
(3) Grant S. (1999) Dissecting the mechanisms of posttranscriptional gene silencing: divide and conquer. Cell 96: 303-306.
(4) Lin et. al. (2001) D-RNAi (messenger RNA-antisense DNA interference) as a novel defense system against cancer and viral infections. Current Cancer Drug Targets 1: 241-247.
(5) Bartel, D. B. (2004) MicroRNAs: Genomics, biogenesis, mechanism and function. Cell 116: 281-297.
(6) Lin et. al. (2004a) Novel RNAi therapy—Intron-derived microRNA drugs. Drug Design Reviews 1: 247-255.
(7) Stark et. al. (1998) How cells respond to interferons. Annu. Rev. Biochem. 67: 227-264.
(8) Brantl S. (2002) Antisense-RNA regulation and RNA interference. Biochimica et Biophysica Acta 1575: 15-25.
(9) Jen et. al. (2000) Suppression of gene expression by targeted disruption of messenger RNA: available opions and current strategies. Stem Cells 18: 307-319.
(10) Ying et. al. (1999) Suppression of activin-induced apoptosis by novel antisense strategy in human prostate cancer cells. Biochem Biophys Res Commun. 265: 669-673.
(11) Tuschl et. al. (2002) Expanding small RNA interference. Nat Biotechnol. 20: 446-448.

(12) Miyagishi et. al. (2002) U6 promoter-driven siRNAs with four uridine 3′ overhangs efficiently suppress targeted gene expression in mammalian cells. Nat Biotechnol 20: 497-500.
(13) Lee et. al. (2002) Expression of small interfering RNAs targeted against HIV-1 rev transcripts in human cells. Nat Biotechnol 20: 500-505.
(14) Paul et. al. (2002) Effective expression of small interfering RNA in human cells. Nat Biotechnol 20: 505-508.
(15) Gunnery et. al. (1995) Functional mRNA can be generated by RNA polymerase III. Mol Cell Biol. 15: 3597-3607.
(16) Schramm et. al. (2002) Recruitment of RNA polymerase III to its target promoters. Genes Dev 16: 2593-2620.
(17) Sledz et. al. (2003) Activation of the interferon system by short interfering RNAs. Nat Cell Biol. 5: 834-839.
(18) Lin et al. (2004b) Combinational Therapy for HIV-1 eradication and vaccination. Intrn'l J. Oncol. 24: 81-88.
(19) Clement et. al. (1999) The stability and fate of a spliced intron from vertebrate cells. RNA 5: 206-220.
(20) Nott et. al. (2003) A quantitative analysis of intron effects on mammalian gene expression. RNA 9: 607-617.
(21) Lin et. al. (2003) A novel RNA splicing-mediated gene silencing mechanism potential for genome evolution. Biochem Biophys Res Commun 310:754-760.
(22) Ghosh et. al. (2000) Coupled in vitro synthesis and splicing of RNA polymerase II transcripts. RNA 6: 1325-1334.
(23) Lin et. al. (2005) Asymmetry of intronic pre-microRNA structures in functional RISC assembly. Gene 356: 32-38.
(24) Holen et. al. (2002) Positional effects of short interfering RNAs targeting the human coagulation trigger tissue factor. Nucleic Acid Res. 30: 1757-1766.
(25) Krol et. al. (2004) Structural features of microRNA (miRNA) precursors and their relevance to miRNA biogenesis and small Interfering RNA/short hairpin RNA design. J. Biol. Chem. 279: 42230-42239.
(26) Rose, A. B. (2002) Requirements for intron-mediated enhancement of gene expression in Arabidopsis. RNA 8: 1444-1451.
(27) Tang, G. (2005) siRNA and miRNA: an insight into RISCs. Trends Biochem Sci. 30: 106-114.
(28) Ying et. al. (2004) Intron-derived microRNAs—fine tuning of gene functions. Gene 342: 25-28.
(29) Jin et al. (2004) RNA and microRNAs in fragile X mental retardation. Nat Cell Biol. 6: 1048-1053.
(30) U.S. Pat. No. 6,066,500 to Bennett.
(31) U.S. Pat. No. 6,506,599 to Fire.
(32) U.S. Pat. No. 4,289,850 to Robinson.
(33) U.S. Pat. No. 6,159,712 to Lau.

It is understood that the examples and embodiments described herein are for illustrative purposes only and that various modifications or changes in light thereof will be suggested to persons skilled in the art, and are to be included within the spirit and purview of the invention as set forth in the appended claims. All publications and patents cited herein are incorporated herein by reference in their entirety for all purposes.

What is claimed is:

1. A method for inducing intron-mediated transgenic gene silencing effects comprises the steps of: (a) Constructing an isolated nucleic acid composition containing at least an intron flanked with a plurality of exons, wherein said intron can be cleaved out of the exons by intracellular RNA splicing and processing mechanisms for triggering gene silencing effects and said exons can be linked together to form a reporter gene transcript with desired function; (b) Introducing said nucleic acid composition into an organism; (c) Generating RNA transcript of said nucleic acid composition in said organism; and (d) Releasing the function of said intron via the RNA splicing- and processing mechanisms, so as to provide gene silencing effects directed against a targeted gene or genes containing at least a sequence complementary to said intron.

2. The method as defined in claim 1, further comprises the step of synthesizing the nucleic acid components of said intron or exon sequences, or both.

3. The method as defined in claim 1, further comprises the step of mixing a plurality of different kinds of said nucleic acid compositions between the step (a) and (b).

4. The method as defined in claim 1, further comprises the step of cloning said nucleic acid composition in an expression-competent vector.

5. The method as defined in claim 1, further comprises the step of mixing a plurality of different kinds of said vectors between the step (b) and (c).

6. The method as defined in claims 4 and 5, wherein said vector is a gene expression-competent vector selected from the group consisting of promoter-linked gene homologue, plasmid, cosmid, phagmid, yeast artificial chromosome, bacteriophage, transposon, retrotransposon, jumping gene, viral vector, and a combination thereof.

7. The method as defined in claims 4 and 5, wherein said vector contains at least a viral or type-II RNA polymerase (Pol-II) promoter, or both, a Kozak consensus translation initiation site, polyadenylation signals and restriction/cloning sites.

8. The method as defined in claims 4 and 5, wherein said vector further contains a pUC origin of replication, a SV40 early promoter for expressing at least an antibiotic resistance gene in replication-competent prokaryotic cells and an optional SV40 origin for replication in eukaryotic cells.

9. The method as defined in claim 8, wherein said antibiotic resistance gene is selected from the group consisted of G418, penicillin G, ampcillin, neomycin, paromycin, kanamycin, streptomycin, erythromycin, spectromycin, phophomycin, tetracycline, rifapicin, amphotericin B, gentamicin, chloramphenicol, cephalothin, tylosin, and a combination thereof.

10. The method as defined in claim 1, wherein said nucleic acid composition is an artificial gene made by DNA ligation.

11. The method as defined in claim 1, wherein said nucleic acid composition is a cellular gene made by the integration of said intron in its sequence.

12. The method as defined in claim 11, wherein said cellular gene is a gene selected from the group consisting of viral gene, bacterial gene, insect gene, plant gene, animal gene, mutated gene, jumping gene, protein-coding as well as non-protein-coding gene, functional as well as non-functional gene, and a combination thereof.

13. The method as defined in claim 11, wherein said intron is integrated into said cellular gene by a gene-engineering method selected from the group consisting of homologous gene recombination, DNA insertion, DNA ligation, transposon insertion, jumping gene integration, electrofusion, retrotransposon fusion, retroviral infection, and a combination thereof.

14. The method as defined in claim 1, wherein said intron is a nucleic acid sequence containing components selected from the group consisting of intronic nucleotide insert, branch point, poly-pyrimidine tract, splicing donor site, splicing acceptor site, and a combination thereof.

15. The method as defined in claim 14, wherein said intronic nucleotide insert is a nucleic acid sequence containing components and/or analogs either homologous or complementary, or both, to a targeted gene or genes selected from the group consisting of pathogenic nucleic acids, viral genes, bacterial genes, diseased genes, dysfunctional genes, mutated genes, oncogenes, jumping genes, transposons, microRNA genes, protein-coding as well as non-protein-coding genes, functional as well as non-functional genes, and a combination thereof.

16. The method as defined in claim 14, wherein said intronic nucleotide insert is a nucleic acid template encoding functional RNA selected from the group consisting of lariat-form RNA, short-temporary RNA (stRNA), antisense RNA, small-interfering RNA (siRNA), double-stranded RNA (dsRNA), short-hairpin RNA (shRNA), microRNA (miRNA), tiny non-coding RNA (tncRNA), snRNA, snoRNA, aberrant RNA containing mismatched base pairing, deoxynucleotidylated RNA (D-RNA), ribozyme RNA and their precursors as well as derivatives in either sense or antisense, or both, orientation, and a combination thereof.

17. The method as defined in claim 14, wherein said intronic nucleotide insert is a sense-oriented nucleic acid sequence containing about 40% to 100% homology to a targeted gene, most preferably containing about 90% to 100% homology to the targeted gene.

18. The method as defined in claim 14, wherein said intronic nucleotide insert is an antisense-oriented nucleic acid sequence containing about 40% to 100% complementarity to a targeted gene, most preferably containing about 90% to 100% complementarity to the targeted gene.

19. The method as defined in claim 14, wherein said intronic nucleotide insert is a hairpin-like nucleic acid sequence containing about 35% to 65% homology and/or about 35% to 65% complementarity to a targeted gene, most preferably containing about 41% to 49% homology and about 41% to 49% complementarity to the targeted gene.

20. The method as defined in claim 14, wherein said intronic nucleotide insert is incorporated into said intron through at least a restriction/cloning site selected from the group consisting of AatII, AccI, AflII/III, AgeI, ApaI/LI, AseI, Asp718I, BamHI, BbeI, BcI/II, BglII, BsmI, Bsp120I, BspHI/LU11I/120I, BsrI/BI/GI, BssHII/SI, BstBI/UI/XI, ClaI, Csp6I, DpnI, DraI/II, EagI, EclI36II, EcoRI/RII/47III, EheI, FspI, HaeIII, HhaI, HinPI, HindIII, HinfI, HpaI/II, KasI, KpnI, MaeII/III, MfeI, MluI, MscI, MseI, NaeI, NarI, NcoI, NdeI, NgoMI, NotI, NruI, NsiI, PmlI, Ppu10I, PstI, PvuI/II, RsaI, SacI/II, SalI, Sau3AI, SmaI, SnaBI, SphI, SspI, StuI, TaiI, TaqI, XbaI, XhoI, XmaI cleavage site, and a combination thereof.

21. The method as defined in claim 14, wherein said branch point is an adenosine (A) nucleotide located within a nucleic acid sequence containing or homologous to the motif of 5′-TACTWAY-3′ sequences (SEQ.ID.NO.3).

22. The method as defined in claim 21, wherein said branch point is an adenosine (A) nucleotide located within a nucleic acid sequence containing at least an oligonucleotide motif homologous to 5′-TACTAAC-3′ or 5′-TACTTATC-3′.

23. The method as defined in claim 14, wherein said poly-pyrimidine tract is a high T or C content oligonucleotide sequence containing or homologous to an oligonucleotide selected from the group consisting of 5′-(TY)m(C/-)(T)nC(C/-)-3′ and 5′-(TC)nNCTAG(G/-)-3′, while the symbols of “m” and “n” indicates multiple repeats ≧1; most preferably, the m number is equal to 1˜3 and the n number is equal to 7˜12.

24. The method as defined in claim 14, wherein said splicing donor site is a nucleic acid sequence either containing or homologous to the 5′-GTAAGAGK-3′ sequences (SEQ.ID.NO.1).

25. The method as defined in claim 24, wherein said splicing donor site is a nucleic acid sequence containing or homologous to 5′-AG GTAAGAGGAT-3′, 5′-AG GTAAGAGT-3′, 5′-AG GTAGAGT-3′ or 5′-AG GTAAGT-3′.

26. The method as defined in claim 14, wherein said splicing acceptor site is a nucleic acid sequence either containing or homologous to the GWKSCYRCAG sequences (SEQ.ID.NO.2).

27. The method as defined in claim 26, wherein said splicing acceptor site is a nucleic acid sequence containing or homologous to 5′-GATATCCTGCAG G-3′, 5′-GGCTGCAG G-3′ or 5′-CCACAG C-3′.

28. The method as defined in claim 1, wherein said nucleic acid composition is introduced into said organism by a gene delivery method selected from the group consisting of liposomal transfection, chemical transfection, chemical transformation, electroporation, homologous recombination, transposon insertion, jumping gene transfection, viral infection, micro-injection, gene-gun penetration, and a combination thereof.

29. The method as defined in claim 1, wherein said organism is selected from the group consisting of microbe, cell, tissue, organ, plant, animal, and a combination thereof.

30. The method as defined in claim 29, wherein said cell is selected from the group consisting of microbe, bacteria, algae, ameba, yeast, cell line, blood cell, and a combination thereof.

31. The method as defined in claim 29, wherein said plant is selected from the group consisting of algae, weed, rice, wheat, flower, fruit, tree and a combination thereof.

32. The method as defined in claim 29, wherein said animal is selected from the group consisting of ameba, parasite, worm, insect, avian, vertebrate, mammal, primate, human, and their derivative tiisues and organs.

33. The method as defined in claim 1, wherein said RNA transcript of the nucleic acid composition is an ribonucleotide sequence selected from the group consisting of mRNA, hnRNA, rRNA, TRNA, snoRNA, snRNA, microRNA, viral RNA and their RNA precursors as well as derivatives in either sense, antisense or both orientations, and a combination thereof.

34. The method as defined in claim 1, wherein said RNA transcript of the nucleic acid composition is generated by transcription machinery selected from the group consisting of type-II (Pol-II), type-I (Pol-I), type-III (Pol-III), type-IV (Pol-IV) and viral RNA polymerase transcription machineries, and a combination thereof.

35. The method as defined in claim 1, wherein said function of the intron is related to the gene silencing activity of an RNA selected from the group consisting of lariat-form RNA, microRNA (miRNA), short-temporary RNA (stRNA), antisense RNA, small-interfering RNA (siRNA), double-stranded RNA (dsRNA), short-hairpin RNA (shRNA), tiny non-coding RNA (tncRNA), snRNA, aberrant RNA containing mismatched base pairing, deoxynucleotidylated RNA (D-RNA), ribozyme RNA and their precursors as well as derivatives, and a combination thereof.

36. The method as defined in claim 1, wherein said function of the intron is released from said intron by an RNA processing mechanism selected from the group consisting of RNA splicing, RNA processing, RNaseIII excision, homologous complementing and repairing, intron-mediated RNA degradation (IME), and a combination thereof.

37. The method as defined in claim 1, wherein said gene silencing effect is caused by an intracellular mechanism selected from the group consisting of RNA interference (RNAi), posttranscriptional gene silencing (PTGS), RNAi-induced transcriptional gene silencing (RITS), co-suppression, quelling, ribozyme-associated RNA degradation, nonsense-mediated degradation (NMD), intron-mediated enhancement (IME), antisense- or microRNA-mediated translation suppression, gene replacement, homologous complementing and repairing mechanisms, and a combination thereof.

38. The gene silencing effect as defined in claim 37, where in said gene silencing effect suppresses the function of a targeted gene selected from the group consisting of GFP, luciferase, lac-Z, integrin, β-catenin, tyrosinase, melanin, FMRP, HIV, HBV, HCV, HPV, flu and their derivatives as well as the combination thereof.

39. The method as defined in claim 1, wherein the desired gene function of said exons is result from a genetic activity selected from the group consisting of normal gene expression, missing gene replacement, dominant-negative gene suppression, siRNA duplex formation, gene marker formation and targeting such as expression of fluorescent protein (GFP), luciferase, lac-Z, and the derivatives as well as a combination thereof.

40. A method of generating an transgenic organism by suppressing gene function or silencing gene expression using an isolated nucleic acid composition, comprising the steps of: a) providing: i) a substrate expressing a targeted gene, and ii) a nucleic acid composition comprising a recombinant gene capable of producing RNA transcript, which is in turn able to generate pre-designed gene silencing molecules through intracellular RNA splicing and/or processing mechanisms to inhibit the targeted gene expression or suppress the targeted gene function in the substrate; b) treating the substrate with the nucleic acid composition under conditions such that the targeted gene expression or function in the substrate is inhibited.

41. The method as defined in claim 40, wherein said substrate is an organism selected from the group consisting of microbe, cell, tissue explant, organ culture, plant, animal, and a combination thereof.

42. The method as defined in claim 40, wherein said targeted gene is selected from the group consisting of pathogenic nucleic acid, viral gene, bacterial gene, diseased gene, dysfunction gene, mutated gene, oncogene, jumping gene, transposon, microRNA gene, protein-coding gene as well as non-protein-coding gene, functional as well as non-functional gene, and a combination thereof.

43. The method as defined in claim 40, where in said targeted gene is selected from the group consisting of GFP, luciferase, lac-Z, integrin, β-catenin, tyrosinase, melanin, FMRP, HIV, HBV, HCV, HPV, flu and their derivatives as well as a combination thereof.

44. The method as defined in claim 40, wherein said nucleic acid composition is an expression-competent nucleic acid vector selected from the group consisting of cellular gene, plasmid, cosmid, phagmid, yeast artificial chromosome, transposon, jumping gene, viral vector, and a combination thereof.

45. The method as defined in claim 44, wherein said vector further contains a viral or type-II RNA polymerase (Pol-II) promoter, or both, a Kozak consensus translation initiation site, polyadenylation signals and restriction/cloning sites.

46. The method as defined in claim 44, wherein said vector further contains a pUC origin of replication, a SV40 early promoter for expressing at least an antibiotic resistance gene in replication-competent prokaryotic cells and an optional SV40 origin for replication in eukaryotic cells.

47. The method as defined in claim 40, wherein said nucleic acid composition comprises a recombinant gene containing at least an intron flanked with a plurality of exons, wherein said intron can be cleaved out of the exons of the recombinant gene via intracellular RNA splicing and/or processing mechanisms for triggering gene silencing effects and said exons can be linked together to form a reporter gene transcript with a desired function.

48. The nucleic acid composition of claim 47, wherein said recombinant gene possesses at least a function selected from the group consisting of normal gene activity, missing gene replacement, dominant-negative gene suppression, RNA duplex formation, reporter gene marker and indicator such as expression of fluorescent protein (GFP), luciferase, lac-Z, and their derivatives as well as a combination thereof.

49. The nucleic acid composition of claim 47, wherein said intron contains a splice donor site that includes 5′-GUA(A/-)GAG(G/U)-3′ or 5′-GU(A/G)AGU-3′, a splice acceptor site that includes 5′-G(A/U/-)(U/G)(C/G)C(U/C)(G/A)CAG-3′ or 5′-CU(A/G)A(C/U)NG-3′, a branch site that includes 5′-UACU(A/U)A(C/U)(-/C)-3′, a poly-pyrimidine tract that includes 5′-(U(C/U))₁₋₃(C/-)U₇₋₁₂C(C/-)-3′ or 5′-(UC)₇₋₁₂NCUAG(G/-)-3′, and a combination thereof.

50. The method as defined in claim 40, wherein said RNA splicing and/or processing mechanism is an intracellular mechanism selected from the group consisting of RNA interference (RNAi), posttranscriptional gene silencing (PTGS), RNaseII excision, RNAi-induced transcriptional gene silencing (RITS), co-suppression, quelling, ribozyme-associated RNA degradation, nonsense-mediated degradation (NMD), intron-mediated enhancement (IME), antisense- or microRNA-mediated translation suppression, gene replacement, rRNA processing, homologous complementing and repairing mechanisms, and a combination thereof.

51. The method as defined in claim 40, wherein said RNA transcript is an RNA selected from the group consisting of mRNA, hnRNA, rRNA, tRNA, snoRNA, snRNA, tncRNA, microRNA, viral RNA, and their precursors as well as derivatives, and a combination thereof.

52. The method as defined in claim 40, wherein said pre-designed gene silencing molecule is an RNA selected from the group consisting of microRNA (miRNA), lariat-form RNA, short-temporary RNA (stRNA), antisense RNA, small-interfering RNA (siRNA), double-stranded RNA (dsRNA), short-hairpin RNA (shRNA), tiny non-coding RNA (tncRNA), aberrant RNA containing mismatched base pairing, deoxynucleotidylated RNA (D-RNA), ribozyme RNA, and their precursors as well as derivatives, and a combination thereof.

53. The method as defined in claim 40, wherein said condition is a transgenic method selected from the group consisting of liposomal transfection, chemical transfection, chemical transformation, electroporation, homologous DNA recombination, DNA insertion, transposon insertion, jumping gene transfection, viral infection, micro-injection, gene-gun penetration, and a combination thereof.
